SEQUENCE LISTING (79 sequences)

SEQ ID NO: 1 - DNA, 507 bp, Artificial Sequence
  Homology regions of 5-prime region of pga genes with flanking BsaI sites
ggtctcagagaataattcctatcagatttttgtcagcgttttaagtatgcgctttcaaaattcgacattatgatttttgtacttttctccctatttcagatgaaaggttgaaggatggattcaggatctcttttttcccccggccgcaatcccctttaaatgggctgtcagccgggatgtctcttttcttttagtattttcgctcttttcgaacaggttaaaaagtcaccaatgttccctcttcaaaacaaatccgtcctatgacaatcagaccatctatcaaaaaatgaaaatctcttttagcataaagcgggattcattgacaaatccaaatattttgtgagtcgttcgatccaccttatttcttcagattttaggttaaaatcctaggatttaaaaagccattaatttttttcaatgataattcatcaaactttaacatttttaatttttcttactattctcattgttactgtattcgccattaaacgcccctctcatgagacc

SEQ ID NO: 2 - DNA, 560 bp, Artificial Sequence
  Homology regions of 3-prime region of pga genes with flanking BsaI sites
ggtctcaacccagttaaaaataaagacatccaaagcaccgcaattccacaaaacaaaaccaactttttgtttgccgctttttttatcaattgatctgttcctcctcttctttcctctttcatcgtcattcaactgggatatttaaaggactttttttgcatgtacagcagcgtcatagcgagaagaaacgctcagcacagcatgatcagctttttcacctaagcgcggattaaaaagatgaagggaaatattctaaagaggtcatgaacatgaggtgcggttcccatcccgaattggtaaggtgatgcattttcaaattttgcttcagtcgaaatttgtcagtttttattcccttcaaaagaatacaataaacatgaaccccaaaaccatgtatatatcctcctgattttctataaaactttatacactgaccggttaagaaaattgtcgtgtttccctttcaaaccggccgcttttcacaggtcttttataaccgggatcagcacacaaaaaagagagcgccggcggcactctcttttgcactgttatgcgacgagacc

SEQ ID NO: 3 - DNA, 110 bp, Artificial Sequence
  Promoter synthetic promoter Psy flanked by type-II restriction sites
atatatgaagactttcgagagtaataagtatcttcatatctattgacaactgaagatatattctatataatcgttctagaaaaggaggtagaatatttgtcttcatatat

SEQ ID NO: 4 - DNA, 218 bp, Bacillus licheniformis
  misc_feature (1)..(218): Functional fragment degQ gene with 3-prime region flanked by type-II restriction sites
gaagacctatatggaaaagcaacaaattgaagaattaaaacaactgctttggcggctagagaatgaaatcagagaaacaaaggactccttgcgcaagattaacaaaagcattgatcaatacgataagtacacatatctaaaaacctcgtaaaaagacttgtttcagacaagtcttttttttgtgtgcaaacgaacccaaaaaaaggtagcaagtcttc

SEQ ID NO: 5 - DNA, 524 bp, Artificial Sequence
  Homology regions of 5-prime region of the CAT gene with flanking BsaI sites
ggtctcagagaacctcatcctgtattgtaaatgaattgtagcgcgtttcggaggccgaagcaaccgatccctccttctttgtattcttgctgtgccgttctccgtctctcacccccggctgtcatttaggttatactgacatatatcggggtgaagatatgaacagcaaatcacgattgacgcctgaaatgtggcgggcaattacggaaaacgattccgcctatgacggagttttttattacgcggtcaaaacgaccggcatattttgccgcccatcctgcaaatcgagagttccgcaaatcgacaatgtgcagatttttttcaatgcaaaagatgctttatcagaagggttccgcccctgcaaacgctgcaatcccgccggagcgctgctgccggatgaagagctggcacagcgggtagtgcaaatcatcgagaaatcttatcgcgatacgctgtctctgcaagctttggctgacaggtgccatatcagcccttttcacctgcagcggacatctcatgagacc

SEQ ID NO: 6 - DNA, 424 bp, Artificial Sequence
  Homology regions of 3-prime region of the CAT gene with flanking BsaI sites
ggtctcaacccaatcctatgtataccggcttttcactccggcttccggcagggtaaataaaaggattggagtgatcaagcaatgccgcaaaacaataacaatcgatatgaactctttttcaaaaaaagatacaaagttctacatatattaaacgattttctcatcggccttttattccttgtcgggagcttctgctttttctccgaggaactgaagccggcgggactatggatgtttgtgatcggcagctttcagctgttaatcaggccgacgatccgtttggttcatgattttcactacagaaagtttgttgagagccggcgttccaaatgccgggagtaaacacaccacagtaagaccgctaagcagcgcatataaaaaaggccgaatcatatcggcctttcctctgtcatgcgacgagacc

SEQ ID NO: 7 - DNA, 23 bp, Artificial Sequence
  Oligonucleotide PCR-amplification of genomic region of CAT gene
actttcttgaagattcagaattc

SEQ ID NO: 8 - DNA, 22 bp, Artificial Sequence
  Oligonucleotide PCR-amplification of genomic region of CAT gene
gatatcggaataggagcaagtg

SEQ ID NO: 9 - DNA, 32 bp, Artificial Sequence
  Oligonucleotide PCR-amplification of pE194
tatatacagctggattcacaaaaaataggcac

SEQ ID NO: 10 - DNA, 33 bp, Artificial Sequence
  Oligonucleotide PCR-amplification of pE194
tatatacagctggattatgtcttttgcgcagtc

SEQ ID NO: 11 - DNA, 30 bp, Artificial Sequence
  Oligonucleotide PCR-amplification of functional mRFP cassette
tatatggatccgtaatcagggtatcgaggc

SEQ ID NO: 12 - DNA, 31 bp, Artificial Sequence
  Oligonucleotide PCR-amplification of functional mRFP cassette
tatatggatccctcattaggcgggctactaa

SEQ ID NO: 13 - DNA, 2633 bp, Bacillus licheniformis
  misc_feature (1)..(2633): Restriction Modification System (RMS) B. licheniformis
tttttgcctacttatttatttgttttcattttcaaattcatcataagaacccatttcacaaaaatcaatggagtaatgttgttcgctgtgtttataaacaattactgagtcaatatttagtctttccagcatttcataggttagaagttctttttcctctaagttcataacttgtcttagtagccattctccaagagcactatttggattagacataagtgctttactattgtcttggcataccttggctgataaaagcgatttgtctggcaagcgtaactgaaaaggtttatcacgagctgggaaaaatgttgggaatacattatgaatccattttggaattggtatataaatctcgttagggtttcgtggtcgacctaaagcattccattggtttagaccgcttttttctggtacatgacgctttgagccacggtctgaaaagagtggaagaataacgtgctcaaggttttcaaaaggattgacagttggtgctggaatttttggaatttcaaagccaaatagtttagccaattcatgataaggattttctaagatttcaacattaatttcttcaataggtttatcagtgataaaacgcttataaagggtgctcttagtgacattaaagctgtattcgtgtagaccgtcttcaaaggtgattgtatttctgttgttacttactttcacatttgtaattgaggagatttcaaccaagtccattggctcttcaaaaataagaattttccctggctttcttgttacacagtggtatatcattgaatcaataccatatgttcttttagtaaattcaattctctcgttacggagagaagcaaccgtgtttattagctcttttggagatttcccacgatacaagtctgagtctttattgaattcagctactttttgaagagtatgaccattaccatgaagaaaagtcttaataccaattccgacacgatttaatgaagcgtcagcagaacagtctgacctccccaagttttcagctccaaatgcttcacaaaaagcattttccacattccttgagaccaaataaggcgagtcactttcagagaacaaattggatagcgaaccagttgagcggagcatttgtttgtatgtagtgcagttgatggctggttgattagtatagaacattatttttcctcctcttttatgcttgtcatttcttctttcagacccaaaaggtagtcagctgatacgttcaatgtttcagctattcttttgaaagtgtccaatgatggagttctattttcactttcatatagtgaccaagtgcttctagtgaccccgactttttcagcgatttggctgggtaataacctacgagcttctcttgcattttgaatacgatttccaaggaaaggtatcatttttgcacctccaagatttgttgttttcagagtatcaccagaacccccgaaaatagtccaaagttagctaacagcaaacaaataaaaataaataagttgtttactcttagcaaacttgttactaaaatttgataaagttattcatttaatccagctcttatgctaaaattgcattagcggacaagcttaatgtttgcaaggaggtataattttgacttatcgagtaggtagtatgtttgctgggataggtggaacttgtttagggtttatccaagctggcgctaggattgtctgggcaaatgaaatagacaaaaatgcttgtattacttatagaaattattttggggatgcttacttacaagagggtgacattaacctaatagataaaaactccatacctgaactggacattttgattggaggttttccttgccaagccttctctatagctggctatcgtaaagggtttgaagatgaaaggggaaacgtgttctttcaaatattagaggtattggaagcacaaagaaatgtttatggacacttaccccaagcaataatgcttgagaatgtaaagaacttatttacacatgatagaggtaatacgtacagagtaataaaagaggctttggaagcctttggttataccgtaaaagctgaggttcttaattcaatggaatacggtaacgtgccacaaaacagagagcggatttatattgtaggttttcaagatgagagccaagctgaaaggtttagctttccagacccaattcctttaacaaatcaacttaatgatgtaattgaccgaactcggagagttgataaaagatattattatgatgaaacctctcaatattatgatatgttgcgagaagccatggacagtacagatacaacttatcaaataagacgtatatatgttcgagaaaatagaagcaatgtttgtcctacactgacagcgaatatgggaactggagggcataatgttcctattgtattagactttgaaaataatataagaaaactaacaccagaagaatgcttactattgcaaggtttcccagctgactatcattttccagaaggcatggcaaacactcacaaatataaacaagctggtaactctgttacggtgccagttataagaagaattgccactaatattattagcgtattgaacattggaatgaatataaatcaagaacatgaatatgcaatagctgaataagaccaactaat

SEQ ID NO: 14 - DNA, 1146 bp, Bacillus licheniformis
  CDS (1)..(1146): CDS of Restrictase B. licheniformis
atgttctatactaatcaaccagccatcaactgcactacatacaaacaaatgctccgctcaactggttcgctatccaatttgttctctgaaagtgactcgccttatttggtctcaaggaatgtggaaaatgctttttgtgaagcatttggagctgaaaacttggggaggtcagactgttctgctgacgcttcattaaatcgtgtcggaattggtattaagacttttcttcatggtaatggtcatactcttcaaaaagtagctgaattcaataaagactcagacttgtatcgtgggaaatctccaaaagagctaataaacacggttgcttctctccgtaacgagagaattgaatttactaaaagaacatatggtattgattcaatgatataccactgtgtaacaagaaagccagggaaaattcttatttttgaagagccaatggacttggttgaaatctcctcaattacaaatgtgaaagtaagtaacaacagaaatacaatcacctttgaagacggtctacacgaatacagctttaatgtcactaagagcaccctttataagcgttttatcactgataaacctattgaagaaattaatgttgaaatcttagaaaatccttatcatgaattggctaaactatttggctttgaaattccaaaaattccagcaccaactgtcaatccttttgaaaaccttgagcacgttattcttccactcttttcagaccgtggctcaaagcgtcatgtaccagaaaaaagcggtctaaaccaatggaatgctttaggtcgaccacgaaaccctaacgagatttatataccaattccaaaatggattcataatgtattcccaacatttttcccagctcgtgataaaccttttcagttacgcttgccagacaaatcgcttttatcagccaaggtatgccaagacaatagtaaagcacttatgtctaatccaaatagtgctcttggagaatggctactaagacaagttatgaacttagaggaaaaagaacttctaacctatgaaatgctggaaagactaaatattgactcagtaattgtttataaacacagcgaacaacattactccattgatttttgtgaaatgggttcttatgatgaatttgaaaatgaaaacaaataa

SEQ ID NO: 15 - protein, 381 aa, Bacillus licheniformis
MFYTNQPAINCTTYKQMLRSTGSLSNLFSESDSPYLVSRNVENAFCEAFGAENLGRSDCSADASLNRVGIGIKTFLHGNGHTLQKVAEFNKDSDLYRGKSPKELINTVASLRNERIEFTKRTYGIDSMIYHCVTRKPGKILIFEEPMDLVEISSITNVKVSNNRNTITFEDGLHEYSFNVTKSTLYKRFITDKPIEEINVEILENPYHELAKLFGFEIPKIPAPTVNPFENLEHVILPLFSDRGSKRHVPEKSGLNQWNALGRPRNPNEIYIPIPKWIHNVFPTFFPARDKPFQLRLPDKSLLSAKVCQDNSKALMSNPNSALGEWLLRQVMNLEEKELLTYEMLERLNIDSVIVYKHSEQHYSIDFCEMGSYDEFENENK

SEQ ID NO: 16 - DNA, 1022 bp, Artificial Sequence
  Homology regions of Fusion of 5-prime and 3-prime regions of restrictase gene with flanking BsaI sites
ggtctcgacccaacttctataaatgtaacgatacgatttattgtttcattaaagtcttcctttatttcatgttcccatattcttttaatgttccaatccttttcctcgtaatatttattaacttccttatctctttttttatttctttcgagttttttctcccaatattccgtattactttttggtatattcccgtgtttttcacacgcatgccagaaacaagaatcaatgaatatgactattttatatttctgtattactatatctggactaccgtataatttcttaacattttttcggaatcttattccacggtgccatagttctttagtaaccttatcttctaattttgaacgagatttgattgcctgcatgttttttcttctttgttcttttgaaaccgtgtcagtcatagaagagtcctccaaagccacaataattgtattctataaacgaggaagcaagccctcaagcttaccccctcttagttccttttttgcctacttatttatatttttcctcctcttttatgcttgtcatttcttctttcagacccaaaaggtagtcagctgatacgttcaatgtttcagctattcttttgaaagtgtccaatgatggagttctattttcactttcatatagtgaccaagtgcttctagtgaccccgactttttcagcgatttggctgggtaataacctacgagcttctcttgcattttgaatacgatttccaaggaaaggtatcatttttgcacctccaagatttgttgttttcagagtatcaccagaacccccgaaaatagtccaaagttagctaacagcaaacaaataaaaataaataagttgtttactcttagcaaacttgttactaaaatttgataaagttattcatttaatccagctcttatgctaaaattgcattagcggacaagcttaatgtttgcaaggaggtataattttgacttatcgagtaggtagtatgtttgctgggataggtggaacttgtttaggctcaggagacc

SEQ ID NO: 17 - DNA, 21 bp, Artificial Sequence
  Oligonucleotide PCR-amplification of restrictase genomic region
gacaatccccttttactgacc

SEQ ID NO: 18 - DNA, 24 bp, Artificial Sequence
  Oligonucleotide PCR-amplification of restrictase genomic region
ctatttcatttgcccagacaatcc

SEQ ID NO: 19 - DNA, 1363 bp, Artificial Sequence
  Homology regions of Fusion of 5-prime and 3-prime regions of pga genes with flanking BsaI sites
ggtctcgacccgaacactgaaattttagaccgggctgggatatcgagacatatataggggcgtttaatggcgaatacagtaacaatgagaatagtaagaaaaattaaaaatgttaaagtttgatgaattatcattgaaaaaaattaatggctttttaaatcctaggattttaacctaaaatctgaagaaataaggtggatcgaacgactcacaaaatatttggatttgtcaatgaatcccgctttatgctaaaagagattttcattttttgatagatggtctgattgtcataggacggatttgttttgaagagggaacattggtgactttttaacctgttcgaaaagagcgaaaatactaaaagaaaagagacatcccggctgacagcccatttaaaggggattgcggccgggggaaaaaagagatcctgaatccatccttcaacctttcatctgaaatagggagaaaagtacaaaaatcataatgtcgaattttgaaagcgcatacttaaaacgctgacaaaaatctgataggaattaagaactttcgatttccaaaaatatcaataaaaagataggcattaatgactcgggcgaggtgatctttgtcacggaaaatttcgtcgtcttctgttacataatgccgattgtgatttcatagtgaaccctgatcccggttataaaagacctgtgaaaagcggccggtttgaaagggaaacacgacaattttcttaaccggtcagtgtataaagttttatagaaaatcaggaggatatatacatggttttggggttcatgtttattgtattcttttgaagggaataaaaactgacaaatttcgactgaagcaaaatttgaaaatgcatcaccttaccaattcgggatgggaaccgcacctcatgttcatgacctctttagaatatttcccttcatctttttaatccgcgcttaggtgaaaaagctgatcatgctgtgctgagcgtttcttctcgctatgacgctgctgtacatgcaaaaaaagtcctttaaatatcccagttgaatgacgatgaaagaggaaagaagaggaggaacagatcaattgataaaaaaagcggcaaacaaaaagttggttttgttttgtggaattgcggtgctttggatgtctttatttttaacgaatcataatgatgtacgcgccgatacgatcggcgagaaaatagcggaaactgccagacagcttgagggtgcgaaatacagctacggcggagagaagccgaaaacggggtttgactcgtcaggctttgtgcaatatgtgtttcaatcgctcgatattacgcttccgagaacggtaaaggaacaatcgactcttggctcaggagacc

SEQ ID NO: 20 - DNA, 20 bp, Artificial Sequence
  Oligonucleotide PCR-amplification of pga genomic region
aaagccttctcctctctatt

SEQ ID NO: 21 - DNA, 20 bp, Artificial Sequence
  Oligonucleotide PCR-amplification of pga genomic region
ttcttgaaaaagacaaggtc

SEQ ID NO: 22 - DNA, 1027 bp, Artificial Sequence
  Homology regions of Fusion of 5-prime and 3-prime regions of the aprE gene with flanking BsaI sites
ggtctcgacccgaagttcttttttaacatataggtaaaacaatacgaaaaaaggcgccaagtattgaagaattgcagcagccgcggcatttcccttttcgattgaagcaaaaaacgtatattgaacagtaagcattccaaaaatggaaaatactaaaatcgaacaaatatctgtttttttcttccatatctgacacacatgttgaaaaccgtttttcattgaaacatataacaagagaatgactcccgatgccagaagcctgacagagacaagcgagccggcttcaaccgctcccctttcaaatatgtactgtgcagcgcttcccgataatccccacaatgaagcccctgcaagcaccatcaatacgcctttcacatgagctgatttcatatctttcacccgtttctgtatgcgatatattgcatattttaatagatgatcgacaaggccgcaacctccttcggcaaaaaatgatctcataaaataaatgaatagtattttcataaaatgagctcaataacatattctaacaaatagcatatagaaaaagctagtgtttttagcactagctttttcttcattctgatgaaggttgttcaatattttgaatccgttccatgatcgtcggatggccgtatttaaaaatcttgacgagaaacggcgggtttgcctcgctcagcccggcttttgagagctcttgaaacgtcgaaaccgctgcatcgctgttttgcgtcagttcaatcgcatactggtcagcagctttttcctgatgcctcgaaactgcgttcgtaaatggagacgacgcgaaagagatgacccccatcagcatcagaagaagcggaagtgcggctagatcggattttcctgcaatatgaaggcttcttccatagcggccgatgatccgcttgtacagcttgtcgatcacataaaagacagcaagggataaaagcagatacccgccaagtcctatgtaaacatgcttcatcacatagtgccccatttcgtgcgccatgatgctcaggagacc

SEQ ID NO: 23 - DNA, 21 bp, Artificial Sequence
  Oligonucleotide PCR-amplification of aprE genomic region
ccggttgtcattgatccttta

SEQ ID NO: 24 - DNA, 21 bp, Artificial Sequence
  Oligonucleotide PCR-amplification of aprE genomic region
atcctcctgcaaaaaccgtat

SEQ ID NO: 25 - DNA, 1022 bp, Artificial Sequence
  Homology regions of Fusion of 5-prime and 3-prime regions of sigF gene with flanking BsaI sites
ggtctcgacccaaaatcattcgccttgagcaatcagagcagcgtgcacttgaaacgttgggggtggcgtcatgaaaaatgaaatgaacattcagtttacagcgctcagccaaaatgaatcgtttgcacgggtgacagtcgctgcttttatcgctcagcttgacccgacgatggatgaactgaccgaaattaaaacggtcgtatccgaagcggtcacaaacgcgatcattcacggttatgaaaactcagggcagggaaacgtatatatttccgtcactctcgaggaccatattgtctatttaacgatccgcgacgaaggagtcggcatccctgatcttgaagaagcgcgccagcccctgttcacgacaaagcctgaactcgagcggtcgggaatgggctttacgatcatggaaaatttcatggatgatatttcgatcgactcctcacctgagatgggaaccacaatacacttaacaaagcacttatcaaaaagcaaagcgctttgcaattaatgaggctgctcatgttgcaggcagcctcggatgtccgatgaaaaaccggacgctcttgggagcgttccggttttttttgtgtggtaatttatggtcttttgcgcctttctgaatgataaatgggatgtacttcatactacaactataaccatcatataggaagtgacccagatggaacgtcaagtttttatcagactccgccaccggcttgaggcagatccggatgaattgattttgctcggccatatcgcacaggtagcgggtgaccgcggatacaaagaaaagcttgaacggctgcctatttatcaggtcagcaaagcggatcaaagcatggtggtgctggacgtgatgaaggtgattgaagctgtacataaatcgtttcctgaccttgatgtccaaaccgtcggcggttctgaaaccatcgtggagatccaatatccgaaaaggggtctgtcgcccgtgcttttcatcgccgtctggctgctcttgttcgtcggtgcctcaggagacc

SEQ ID NO: 26 - DNA, 19 bp, Artificial Sequence
  Oligonucleotide PCR-amplification of sigF genomic region
agcagctcggcggagaaat

SEQ ID NO: 27 - DNA, 21 bp, Artificial Sequence
  Oligonucleotide PCR-amplification of sigF genomic region
accttgcccgtcaccatttcg

SEQ ID NO: 28 - DNA, 865 bp, Artificial Sequence
  Functional fragment T2A lacZ cassette
gcgatgttaaggaccgtctcatctcacctgcaaggtctcatctcttttactccatctggatttgttcagaacgctcggttgccgccgggcgttttttatctaaaactagtgtcgagggtcttcggtaccgcgatttacatatgctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgccgcgtttataatgaagaccctagcaggcatcaaataaaacgaaaggctcagtcgaaagactgggcctttcgttttatctgttgtttgtcggtgaacgctctcctgagtaggacaaatccgccgccctagacagctgtcgctgagacgatcgctgagacctcgcaagttctcgccatcgcaggtgaaatctagatgtattcgc

SEQ ID NO: 29 - DNA, 35 bp, Artificial Sequence
  Oligonucleotide PCR-amplification of BLAP expression cassette
ggagaagaccttcgacctcgggacctctttccctc

SEQ ID NO: 30 - DNA, 36 bp, Artificial Sequence
  Oligonucleotide PCR-amplification of BLAP expression cassette
tccgaagacccgctattagcgtgttgccgcttctgc

SEQ ID NO: 31 - DNA, 1055 bp, Artificial Sequence
  Homology regions of Fusion of 5-prime and 3-prime regions of forD gene with flanking BsaI sites
ggtctcgacccggatcccattcggagtatttctgaatgatagagccacacggtccacgttctcactggctaaccggatcaaatgatcttcaggagtcagcataatacatccagttcaggtagataagatttgaatttggtgacttgcttttgttcttcttctttcattttctgactaatccaaactggaaaaagcaggtcttttaacagattaggaggtttctgacatgcaccattcggtcactaaccgaatgcagtaaaggacactgtggtgcttgccagccattagggtattgaggaggtgatcaaaatgctaggtgacagtatttcgtcgaagtggacaagtcgtgaccaaatgacctcggatcgagggttggtcatggaggaaaaaattgatgtctggtgacaaagaggagtcatgatcatggcaccgccaacgagggaaaaaactcttcccgcatcgacacggtatgtgggcggtgacaaactaacttatagagtaaatttattagtcgaatgaaagctcctccatgaaatataatcaaagggaaaacggttgctgtcaacggggctagcatggcaagacccagaaaagttctgggagatcccgctttgcataagcgtattatagtggatgacgcgggctttgttgtttacacttcttgcacctgctgacggcaatcatccctatctatgaaatcgagatttcagcaggccgttattttcgagagagttaaatctatattcattgtttttattttggtaaggacataccggattttaggtttggattaccggtcgagttagcttgtcttttcgcccactaccgtgtcgatgcgggagcaatttaccagaagcacttaccgattgatagttttttattccggtgattgcaaagtttcataaactctgagaattcaataggggtaataccccgctttgaggggcgcggcattttatgcgccccgagtatttattcttaaaatttttaaattaatgtatctatataaaaaggagatgctttcggtgtactgccaaaccatggctcaggagacc

SEQ ID NO: 32 - DNA, 20 bp, Artificial Sequence
  Oligonucleotide PCR-amplification of forD genomic region
ttccggaagctgaaacaccc

SEQ ID NO: 33 - DNA, 20 bp, Artificial Sequence
  Oligonucleotide PCR-amplification of forD genomic region
aacggtctcaccatcaagca

SEQ ID NO: 34 - DNA, 381 bp, Bacillus licheniformis
  CDS (1)..(381): CDS of forD
atgaaaaatcatttgtatgagaaaaaaaagaggaaacctttgactcggacaattaaagcgacgctcgccgtgttgacaatgtccatcgctttggtgggaggcgctacggtgccttcatttgcatgggtgaatccgggttatcactaccagtacccatcggaaggtggtacatggaggtatggattcgtaaacgccgggctccgttcagagtacaaccacccgacaaaggtccacggctcgacagtgcaaaagctcatcgatggaaaagtggataaaacgaatagaagtattgatacggctgcgggccgctactctaatgcctatgtcggagccataaactcacctggtcttaagggtcgttactactatcgcaccaactaa

SEQ ID NO: 35 - protein, 126 aa, Bacillus licheniformis
MKNHLYEKKKRKPLTRTIKATLAVLTMSIALVGGATVPSFAWVNPGYHYQYPSEGGTWRYGFVNAGLRSEYNHPTKVHGSTVQKLIDGKVDKTNRSIDTAAGRYSNAYVGAINSPGLKGRYYYRTN

SEQ ID NO: 36 - protein, 126 aa, Bacillus licheniformis; identical to SEQ ID NO: 35
  MISC_FEATURE (1)..(126): Protein of ForD (Formosin)

SEQ ID NO: 37 - DNA, 141 bp, Bacillus licheniformis
  CDS (1)..(141): CDS of degQ
gtggaaaagcaacaaattgaagaattaaaacaactgctttggcggctagagaatgaaatcagagaaacaaaggactccttgcgcaagattaacaaaagcattgatcaatacgataagtacacatatctaaaaacctcgtaa

SEQ ID NO: 38 - protein, 46 aa, Bacillus licheniformis
VEKQQIEELKQLLWRLENEIRETKDSLRKINKSIDQYDKYTYLKTS

SEQ ID NO: 39 - DNA, 141 bp, Bacillus subtilis
  CDS (1)..(141): CDS of degQ
atggaaaagaaacttgaagaagtaaaacaattgttattccgactcgaacttgatattaaagaaacgacagattcattacgaaacattaacaaaagcattgatcaactcgataaatacaattatgcaatgaaaatttcgtga

SEQ ID NO: 40 - protein, 46 aa, Bacillus subtilis
MEKKLEEVKQLLFRLELDIKETTDSLRNINKSIDQLDKYNYAMKIS

SEQ ID NO: 41 - protein, 46 aa, Bacillus licheniformis
  MISC_FEATURE (1)..(46): Protein of DegQ
MEKQQIEELKQLLWRLENEIRETKDSLRKINKSIDQYDKYTYLKTS

SEQ ID NO: 42 - protein, 46 aa, Bacillus subtilis; identical to SEQ ID NO: 40
  MISC_FEATURE (1)..(46): Protein of DegQ

SEQ ID NO: 43 - DNA, 1158 bp, Bacillus licheniformis
  CDS (1)..(1158): CDS of degS
gtgagcgtttccaaaatggactccaaagttttagattcaatcattatgaagatgttaaaaacggttgacgggagcaaggatgaagtctttcaaatcggagagcagtcccgccagcaatacgaaggcttggtagaagagctgaaacagattaaacagcaggtcaacgaagtcatcgatcttggagacaggctggaagtgcatgcccgccatgcgcggaaccgcttgtcagaggtcagcagaaactttcataaattcagtgaagaagagattcgcgaagcttatgaaaaagcccacaaactgcaggttgaactgacaatgatccagcagcgggaaaagcagctgagagagaagcgtgatgatctggaacggcgccttttggggcttcaggaaatcatcgagcgttcagaagggcttgtcagtcagattaccgtcgtcttaaactacttaaatcaggatctgcgccaggtcggcgttctccttgaagatgcgcaggccaagcaggatttcggactgcggattatcgaagcccaggaggaagaaagaaaaagggtctcaagggaaattcacgacggtcctgcgcaaatgctggccaacgttatgatgagatctgaactgatcgaacgcatcttcagagacaaaggaacagaagaaggctttcaggaaattaaaaacctccggcaaaacgtcagaaatgctctttatgaagtcagaagaatcatttatgatttaaggccgatggctttagacgatttgggattgataccgacgctcagaaaatacttgaacacgatcgaagattatcatggaaaagcaaagattcatttccaatgcatcggagaatccgaagaaagaagaatagcaccgcggtttgaggttgcactattccggcttgcacaggaagcggtgacaaacgccttaaaacactccgaatcaactgaaattcatgttaaagtagaagtgacaaaagattttgtgacgctgattatcaaagacaatggaaacggctttgacttaaaagaagtaaaaggcaagaagaacaaatctttcggtctgctaggtatgaaagaaagagtcgatttgctcgaaggctcaatgacaatcgattcgaaaataggtcttgggacatttatattgattaaagttccactgtctttgtaa

SEQ ID NO: 44 - protein, 385 aa, Bacillus licheniformis
VSVSKMDSKVLDSIIMKMLKTVDGSKDEVFQIGEQSRQQYEGLVEELKQIKQQVNEVIDLGDRLEVHARHARNRLSEVSRNFHKFSEEEIREAYEKAHKLQVELTMIQQREKQLREKRDDLERRLLGLQEIIERSEGLVSQITVVLNYLNQDLRQVGVLLEDAQAKQDFGLRIIEAQEEERKRVSREIHDGPAQMLANVMMRSELIERIFRDKGTEEGFQEIKNLRQNVRNALYEVRRIIYDLRPMALDDLGLIPTLRKYLNTIEDYHGKAKIHFQCIGESEERRIAPRFEVALFRLAQEAVTNALKHSESTEIHVKVEVTKDFVTLIIKDNGNGFDLKEVKGKKNKSFGLLGMKERVDLLEGSMTIDSKIGLGTFILIKVPLSL

SEQ ID NO: 45 - DNA, 1158 bp, Bacillus subtilis
  CDS (1)..(1158): CDS of degS
atgaataaaacaaagatggattccaaagtgctggattctattttgatgaagatgctgaaaaccgttgacgggagcaaggacgaggtttttcaaatcggggagcagtcacgccagcagtatgaacagctggtcgaagaactgaaacaaattaaacagcaggtgtatgaagtgattgagcttggcgataaacttgaagtgcaaactcgccatgcgagaaaccgtttatccgaggtcagccgtaattttcatagattcagtgaagaggaaatccgcaatgcttatgaaaaagcccataagctgcaggtagaattgacgatgatccagcagcgtgagaagcaattgcgcgaacggcgggacgatttggagcgcagattgctagggcttcaggaaatcattgagcggtcagaatcattagtaagccaaattacagttgtgctcaactacttgaatcaggatttgcgcgaagttggactgcttcttgctgatgctcaggcaaaacaggatttcggcttaagaattattgaggcgcaggaagaagagcgaaaaagagtctcaagagaaatccatgacggacccgctcaaatgctggcgaatgttatgatgagatcggaattaatcgagcggattttccgtgaccggggcgcagaggacggattccaagaaattaaaaatctccgccaaaatgttcggaatgccctttacgaagtgagaaggattatatatgatttaagaccgatggcccttgatgacctaggcctgattccaactttaagaaaatatctatatacaaccgaggaatataacgggaaggtcaaaatacattttcagtgcattggagaaacagaggatcagaggctagcgcctcagtttgaggttgcgctcttcaggctcgcacaggaagctgtgtctaatgcgctaaagcattctgaatctgaagaaattacagtcaaagttgagatcacaaaggattttgtgattttaatgataaaagataacggtaaagggttcgacctgaaggaagcgaaagagaagaaaaacaaatcattcggcttgctgggcatgaaagaaagagtagatttattggaaggaacgatgacaatagattcgaaaataggtcttgggacatttattatgattaaggttccgttatctctttga

SEQ ID NO: 46 - protein, 385 aa, Bacillus subtilis
MNKTKMDSKVLDSILMKMLKTVDGSKDEVFQIGEQSRQQYEQLVEELKQIKQQVYEVIELGDKLEVQTRHARNRLSEVSRNFHRFSEEEIRNAYEKAHKLQVELTMIQQREKQLRERRDDLERRLLGLQEIIERSESLVSQITVVLNYLNQDLREVGLLLADAQAKQDFGLRIIEAQEEERKRVSREIHDGPAQMLANVMMRSELIERIFRDRGAEDGFQEIKNLRQNVRNALYEVRRIIYDLRPMALDDLGLIPTLRKYLYTTEEYNGKVKIHFQCIGETEDQRLAPQFEVALFRLAQEAVSNALKHSESEEITVKVEITKDFVILMIKDNGKGFDLKEAKEKKNKSFGLLGMKERVDLLEGTMTIDSKIGLGTFIMIKVPLSL

SEQ ID NO: 47 - protein, 385 aa, Bacillus licheniformis
  MISC_FEATURE (1)..(385): Protein of DegS
MSVSKMDSKVLDSIIMKMLKTVDGSKDEVFQIGEQSRQQYEGLVEELKQIKQQVNEVIDLGDRLEVHARHARNRLSEVSRNFHKFSEEEIREAYEKAHKLQVELTMIQQREKQLREKRDDLERRLLGLQEIIERSEGLVSQITVVLNYLNQDLRQVGVLLEDAQAKQDFGLRIIEAQEEERKRVSREIHDGPAQMLANVMMRSELIERIFRDKGTEEGFQEIKNLRQNVRNALYEVRRIIYDLRPMALDDLGLIPTLRKYLNTIEDYHGKAKIHFQCIGESEERRIAPRFEVALFRLAQEAVTNALKHSESTEIHVKVEVTKDFVTLIIKDNGNGFDLKEVKGKKNKSFGLLGMKERVDLLEGSMTIDSKIGLGTFILIKVPLSL

SEQ ID NO: 48 - protein, 385 aa, Bacillus subtilis; identical to SEQ ID NO: 46
  MISC_FEATURE (1)..(385): Protein of DegS

SEQ ID NO: 49 - protein, 385 aa, Bacillus licheniformis
  MISC_FEATURE (1)..(385): Protein of DegS_S76D
MSVSKMDSKVLDSIIMKMLKTVDGSKDEVFQIGEQSRQQYEGLVEELKQIKQQVNEVIDLGDRLEVHARHARNRLDEVSRNFHKFSEEEIREAYEKAHKLQVELTMIQQREKQLREKRDDLERRLLGLQEIIERSEGLVSQITVVLNYLNQDLRQVGVLLEDAQAKQDFGLRIIEAQEEERKRVSREIHDGPAQMLANVMMRSELIERIFRDKGTEEGFQEIKNLRQNVRNALYEVRRIIYDLRPMALDDLGLIPTLRKYLNTIEDYHGKAKIHFQCIGESEERRIAPRFEVALFRLAQEAVTNALKHSESTEIHVKVEVTKDFVTLIIKDNGNGFDLKEVKGKKNKSFGLLGMKERVDLLEGSMTIDSKIGLGTFILIKVPLSL

SEQ ID NO: 50 - protein, 385 aa, Bacillus subtilis
  MISC_FEATURE (1)..(385): Protein of DegS_S76D
MNKTKMDSKVLDSILMKMLKTVDGSKDEVFQIGEQSRQQYEQLVEELKQIKQQVYEVIELGDKLEVQTRHARNRLDEVSRNFHRFSEEEIRNAYEKAHKLQVELTMIQQREKQLRERRDDLERRLLGLQEIIERSESLVSQITVVLNYLNQDLREVGLLLADAQAKQDFGLRIIEAQEEERKRVSREIHDGPAQMLANVMMRSELIERIFRDRGAEDGFQEIKNLRQNVRNALYEVRRIIYDLRPMALDDLGLIPTLRKYLYTTEEYNGKVKIHFQCIGETEDQRLAPQFEVALFRLAQEAVSNALKHSESEEITVKVEITKDFVILMIKDNGKGFDLKEAKEKKNKSFGLLGMKERVDLLEGTMTIDSKIGLGTFIMIKVPLSL

SEQ ID NO: 51 - protein, 385 aa, Bacillus licheniformis
  MISC_FEATURE (1)..(385): Protein of DegS_G128E
MSVSKMDSKVLDSIIMKMLKTVDGSKDEVFQIGEQSRQQYEGLVEELKQIKQQVNEVIDLGDRLEVHARHARNRLSEVSRNFHKFSEEEIREAYEKAHKLQVELTMIQQREKQLREKRDDLERRLLGLQEIIERSEGLVSQITVVLNYLNQDLRQVGVLLEDAQAKQDFGLRIIEAQEEERKRVSREIHDGPAQMLANVMMRSELIERIFRDKGTEEEFQEIKNLRQNVRNALYEVRRIIYDLRPMALDDLGLIPTLRKYLNTIEDYHGKAKIHFQCIGESEERRIAPRFEVALFRLAQEAVTNALKHSESTEIHVKVEVTKDFVTLIIKDNGNGFDLKEVKGKKNKSFGLLGMKERVDLLEGSMTIDSKIGLGTFILIKVPLSL

SEQ ID NO: 52 - protein, 385 aa, Bacillus subtilis
  MISC_FEATURE (1)..(385): Protein of DegS_G218E
MNKTKMDSKVLDSILMKMLKTVDGSKDEVFQIGEQSRQQYEQLVEELKQIKQQVYEVIELGDKLEVQTRHARNRLSEVSRNFHRFSEEEIRNAYEKAHKLQVELTMIQQREKQLRERRDDLERRLLGLQEIIERSESLVSQITVVLNYLNQDLREVGLLLADAQAKQDFGLRIIEAQEEERKRVSREIHDGPAQMLANVMMRSELIERIFRDRGAEDEFQEIKNLRQNVRNALYEVRRIIYDLRPMALDDLGLIPTLRKYLYTTEEYNGKVKIHFQCIGETEDQRLAPQFEVALFRLAQEAVSNALKHSESEEITVKVEITKDFVILMIKDNGKGFDLKEAKEKKNKSFGLLGMKERVDLLEGTMTIDSKIGLGTFIMIKVPLSL

SEQ ID NO: 53 - DNA, 141 bp, Bacillus pumilus
  CDS (1)..(141): CDS of DegQ Bacillus pumilus strain SH-B9
atggaaaagtatgaaatcgaagaacttaaacaattattatggaaacttgaaaacgaaattagagaaacaacggcttctcttcataacattaacaaaagcattgatcagtacgacaaatatgaatatgtgaaaatttcttaa

SEQ ID NO: 54 - protein, 46 aa, Bacillus pumilus
MEKYEIEELKQLLWKLENEIRETTASLHNINKSIDQYDKYEYVKIS

SEQ ID NO: 55 - DNA, 141 bp, Bacillus velezensis
  CDS (1)..(141): CDS of DegQ Bacillus velezensis FZB42
gtggaaaacaaattagaagaagtaaagcaattattattccgacttgaaaatgatatcagagaaacaaccgactcattacgaaacattaacaaaagcattgatcagctcgataaattctcatatgcaatgaaaatttcttaa

SEQ ID NO: 56 - protein, 46 aa, Bacillus velezensis
VENKLEEVKQLLFRLENDIRETTDSLRNINKSIDQLDKFSYAMKIS

SEQ ID NO: 57 - DNA, 141 bp, Bacillus amyloliquefaciens
  CDS (1)..(141): CDS of DegQ Bacillus amyloliqquefaciens XH7
gtggaaaagaaattagaagaagtaaagcaattattattccgacttgaaaatgatatcagagaaacaaccgactcattacgaaacattaacaaaagcattgatcagctcgataaattctcatatgcaatgaaaatttcttaa

SEQ ID NO: 58 - protein, 46 aa, Bacillus amyloliquefaciens
VEKKLEEVKQLLFRLENDIRETTDSLRNINKSIDQLDKFSYAMKIS

SEQ ID NO: 59 - protein, 46 aa, Bacillus pumilus; identical to SEQ ID NO: 54
  MISC_FEATURE (1)..(46): Protein of DegQ Bacillus pumilus strain SH-B9

SEQ ID NO: 60 - protein, 46 aa, Bacillus velezensis
  MISC_FEATURE (1)..(46): Protein of DegQ Bacillus velezensis FZB42
MENKLEEVKQLLFRLENDIRETTDSLRNINKSIDQLDKFSYAMKIS

SEQ ID NO: 61 - protein, 46 aa, Bacillus amyloliquefaciens
  MISC_FEATURE (1)..(46): Protein of DegQ Bacillus amyloliquefaciens XH7
MEKKLEEVKQLLFRLENDIRETTDSLRNINKSIDQLDKFSYAMKIS

SEQ ID NO: 62 - DNA, 690 bp, Bacillus licheniformis
  CDS (1)..(690): CDS of degU
gtgactaaagtaaatattgtaattattgacgatcatcagttattccgtgaaggtgtcaaacggattttggatttcgaacctacctttgaggtagtggccgaaggagacgacggagatgaagcggctcgcattgtcgagcactaccatcctgatgttgttatcatggatattaatatgccgaatgtgaacggagtagaagcgacaaaacaactggtcgacttgtatccggaatcaaaggttattattttatccatccatgatgacgaaaactatgttacacatgcattaaaaacaggagcccggggctatctgctgaaagaaatggatgccgatacgctgatcgaagccgtgaaagtagtagctgaaggcggatcttatctgcatcctaaggttacacacaatcttgtgaatgaattccgccgtcttgcaacaagcggtgtatcatctcacgctcagcatgaggtgtatccggaaatccggagacctcttcacattctcacaagaagggaatgcgaggtactgcagatgctggcggatggaaaaagcaaccgcggaatcggcgaatcattatttatcagtgaaaaaacggttaaaaaccatgtcagcaacatccttcaaaaaatgaatgtaaacgacagaacgcaggctgttgttgtagccattaaaaacggctgggtagaaatgagataa

SEQ ID NO: 63 - protein, 229 aa, Bacillus licheniformis
VTKVNIVIIDDHQLFREGVKRILDFEPTFEVVAEGDDGDEAARIVEHYHPDVVIMDINMPNVNGVEATKQLVDLYPESKVIILSIHDDENYVTHALKTGARGYLLKEMDADTLIEAVKVVAEGGSYLHPKVTHNLVNEFRRLATSGVSSHAQHEVYPEIRRPLHILTRRECEVLQMLADGKSNRGIGESLFISEKTVKNHVSNILQKMNVNDRTQAVVVAIKNGWVEMR

SEQ ID NO: 64 - protein, 229 aa, Bacillus licheniformis
  MISC_FEATURE (1)..(229): Protein of DegU
MTKVNIVIIDDHQLFREGVKRILDFEPTFEVVAEGDDGDEAARIVEHYHPDVVIMDINMPNVNGVEATKQLVDLYPESKVIILSIHDDENYVTHALKTGARGYLLKEMDADTLIEAVKVVAEGGSYLHPKVTHNLVNEFRRLATSGVSSHAQHEVYPEIRRPLHILTRRECEVLQMLADGKSNRGIGESLFISEKTVKNHVSNILQKMNVNDRTQAVVVAIKNGWVEMR

SEQ ID NO: 65 - DNA, 690 bp, Bacillus subtilis
  CDS (1)..(690): CDS of degU
gtgactaaagtaaacattgttattatcgacgaccatcagttatttcgtgaaggtgttaaacggatattggattttgaacctacctttgaagtggtagccgaaggtgatgacggggacgaagcggctcgtattgttgagcactatcatcctgatgttgtgatcatggatatcaatatgccaaacgtaaatggtgtggaagctacaaaacagcttgtagagctgtatcctgaatctaaagtaattattctatcaattcacgatgacgaaaattatgtaacacatgccctgaaaacaggtgcaagaggttatctgctgaaagagatggatgctgatacattaattgaagcggttaaagtagtggctgagggcggatcttacctccatccgaaggttactcacaacctcgttaacgaattccgccgccttgcaacaagcggagtttctgcacaccctcaacatgaggtttaccctgaaatccgcagaccattacatattttaactaggcgggaatgtgaagtgctgcagatgcttgcagacggaaaaagcaaccgcggtattggtgaatcattgtttatcagtgagaaaaccgttaaaaaccatgtcagcaatattttacaaaaaatgaatgtaaacgaccggacgcaagccgttgtggtcgccattaaaaatggctgggtagaaatgagatag

SEQ ID NO: 66 - protein, 229 aa, Bacillus subtilis
VTKVNIVIIDDHQLFREGVKRILDFEPTFEVVAEGDDGDEAARIVEHYHPDVVIMDINMPNVNGVEATKQLVELYPESKVIILSIHDDENYVTHALKTGARGYLLKEMDADTLIEAVKVVAEGGSYLHPKVTHNLVNEFRRLATSGVSAHPQHEVYPEIRRPLHILTRRECEVLQMLADGKSNRGIGESLFISEKTVKNHVSNILQKMNVNDRTQAVVVAIKNGWVEMR

SEQ ID NO: 67 - protein, 229 aa, Bacillus subtilis
  MISC_FEATURE (1)..(229): Protein of DegU
MTKVNIVIIDDHQLFREGVKRILDFEPTFEVVAEGDDGDEAARIVEHYHPDVVIMDINMPNVNGVEATKQLVELYPESKVIILSIHDDENYVTHALKTGARGYLLKEMDADTLIEAVKVVAEGGSYLHPKVTHNLVNEFRRLATSGVSAHPQHEVYPEIRRPLHILTRRECEVLQMLADGKSNRGIGESLFISEKTVKNHVSNILQKMNVNDRTQAVVVAIKNGWVEMR

SEQ ID NO: 68 - DNA, 183 bp, Bacillus licheniformis
  CDS (1)..(183): CDS of degR
gtggaaagcaatgaactgcaaaccgttcttcacaagacatttgtggaggtatataaagatcttgaacagctcgttgatatcgctaaaaaggggcgtccatctctcgagaagaacattgaggaaatcgagcaaaggctgaagcaaaatatactggcgatcgaaattcagctgaaaattaaatag

SEQ ID NO: 69 - protein, 60 aa, Bacillus licheniformis
VESNELQTVLHKTFVEVYKDLEQLVDIAKKGRPSLEKNIEEIEQRLKQNILAIEIQLKIK

SEQ ID NO: 70 - protein, 60 aa, Bacillus licheniformis
  MISC_FEATURE (1)..(60): Protein of DegR
MESNELQTVLHKTFVEVYKDLEQLVDIAKKGRPSLEKNIEEIEQRLKQNILAIEIQLKIK

SEQ ID NO: 71 - DNA, 183 bp, Bacillus subtilis
  CDS (1)..(183): CDS of degR
atggatgataaagacttgaagttgatccttcacaaaacatttatagaaatatacagtgatttagaagaactggccgatatcgcgaaaaaaggaaaaccatcaatggaaaagtatgttgaagagattgaacagaggtgtaaacaaaacattttggcgattgaaatccagatgaaaatcaaatag

SEQ ID NO: 72 - protein, 60 aa, Bacillus subtilis
MDDKDLKLILHKTFIEIYSDLEELADIAKKGKPSMEKYVEEIEQRCKQNILAIEIQMKIK

SEQ ID NO: 73 - protein, 60 aa, Bacillus subtilis; identical to SEQ ID NO: 72
  MISC_FEATURE (1)..(60): Protein of DegR

SEQ ID NO: 74 - DNA, 117 bp, Bacillus licheniformis
  CDS (1)..(117): CDS of phrG
atgaaaaaactgttcattgttgctgcgattgctgccgtcgtatgttcgggatggtttgcggcagaaactcactgggcatccggcgacatgcaggttgctgaaaagatggtcggttaa

SEQ ID NO: 75 - protein, 38 aa, Bacillus licheniformis
MKKLFIVAAIAAVVCSGWFAAETHWASGDMQVAEKMVG

SEQ ID NO: 76 - protein, 38 aa, Bacillus licheniformis; identical to SEQ ID NO: 75

SEQ ID NO: 77 - DNA, 117 bp, Bacillus subtilis
  CDS (1)..(117): CDS of phrG
atgaaaagatttctgattggcgcaggcgtcgcagcggtgattttatcaggttggtttattgcggaccatcaaacccactcacaggaaatgaaagtcgctgagaaaatgattggataa

SEQ ID NO: 78 - protein, 38 aa, Bacillus subtilis
MKRFLIGAGVAAVILSGWFIADHQTHSQEMKVAEKMIG

SEQ ID NO: 79 - protein, 38 aa, Bacillus subtilis; identical to SEQ ID NO: 78
  MISC_FEATURE (1)..(38): Protein of phrG

(19) United States
(12) Patent Application Publication
(10) Pub. No.: US 2024/0318188 A1
(43) Pub. Date: Sep. 26, 2024

(54) BACILLUS LICHENIFORMIS HOST CELL FOR PRODUCTION OF A COMPOUND OF INTEREST WITH INCREASED PURITY

(71) Applicant: BASF SE, Ludwigshafen (DE)

(72) Inventors: Max Fabian Felle, Ludwigshafen (DE); Mathis Appelbaum, Ludwigshafen (DE); Christopher Sauer, Ludwigshafen (DE); Stefan Jenewein, Ludwigshafen (DE)

(21) Appl. No.: 18/572,827

(22) PCT Filed: Jun. 24, 2022
(86) PCT No.: PCT/EP2022/067439

§ 371 Date: Dec. 21, 2023

(30) Foreign Application Priority Data
Jun. 24, 2021 (EP) 21181349.8

(51) Int. Cl. C12N 15/75; C07K 14/32; C12N 9/48

(52) U.S. Cl. CPC C12N 15/75; C07K 14/32; C12N 9/48

(57) ABSTRACT

The present invention relates to a Bacillus licheniformis host cell for production of biological compounds with increased purity. Specifically, the invention relates to a Bacillus licheniformis host with one or more genetic modifications selected from a) a genetic modification causing compared to an unmodified control cell an increased expression of at least one of the genes selected from the group consisting of degQ, degll, degS, degR, and phrG, b) a genetic modification causing compared to an unmodified control cell an increased autophosphorylation activity of the DegS protein, and c) a genetic modification causing compared to an unmodified control cell a reduced phosphatase activity of the DegS protein. The present invention further relates to a method for production of at least one compound of interest with increased purity based on cultivating the bacterial host cell of the present invention.

### FIELD OF THE INVENTION

The present invention relates to a Bacillus licheniformis host cell for the production of biological compounds with improved purity. Specifically, the invention relates to a Bacillus licheniformis host with one or more genetic modifications selected from
a) a genetic modification causing compared to an unmodified control cell an increased expression of at least one of the genes selected from the group consisting of degQ, degU, degS, degR, and phrG,
b) a genetic modification causing compared to an unmodified control cell an increased autophosphorylation activity of the DegS protein, and
c) a genetic modification causing compared to an unmodified control cell a reduced phosphatase activity of the DegS protein.

The present invention further relates to a method for production of at least one compound of interest with improved purity based on cultivating the bacterial host cell of the present invention.

### BACKGROUND

Microorganisms of the Bacillus genus are widely applied as industrial workhorses for the production of valuable compounds, such as chemicals, polymers and proteins, in particular proteins like washing- and/or cleaning-active enzymes or enzymes used for feed and food applications.

The biotechnological production of these useful substances is conducted via fermentation of such Bacillus species and subsequent purification of the product. Among various Bacillus species, Bacillus licheniformis has been widely used as industrial production host for secreted polymers (WO2005098016) and proteins (WO9102792, WO2016180928).

Gram-positive microorganisms of the genus Bacillus have evolved cellular differentiation, adaptation and survival mechanisms to cope with changing environmental conditions such as heat, drought, water excess, nutrient limitations. In particular, temporal and spatial distinct differentiation of Bacillus cells within a colony provides a survival strategy with individual cells fulfilling different functions like competence development and swarming motility, biofilm formation, production of extracellular degradative enzymes and synthesis antibiotics and spores. These differentiation processes are regulated via complex cell-to-cell signaling networks with integration via intracellular signal transduction cascades finally leading to differentiated gene expression. Among different cellular regulator systems, the DegS-DegU two-component system controls various cellular processes in B. subtilis and related Bacillus species. The response regulator DegU and its cognate sensor kinase DegS control the expression of more than 100 genes (Mäder, U., Homuth, G.; 2002; Molecular Genetics and Genomics, 268(4), 455-467). Hereby the phosphorylation status of DegU as well as the overall level of phosphorylated DegU (DegU-P) within the cell determines differential gene expression (Verhamme, Daniel T.; Nicola R.; 2007; In: Molecular microbiology 65 (2), S. 554-568). In addition to DegS, the phosphorylation status of DegU is affected by several other proteins such as DegQ, DegR, RapG, and PhrG.

Several mutations affecting the DegS-DegU two-component system were mapped to genes encoding components of the DegS/DegU system such as the degU32 (DegU-H12L), degU31 (DegU-V131L), degS100 (DegS V236M), degS200 (DegS-G218E), and degS-S76D (DegS-S76D) (Amory, A., Kunst, F., Aubert, E., Klier, A., & Rapoport, G; (1987); Journal of bacteriology, 169(1), 324-333.; Msadek T, Dedonder R; 1990; J Bacteriol 172(2):824-834). The overexpression of a degQ gene has been described in Bacillus subtilis to enhance extracellular enzyme production (U.S. Pat. Nos. 5,017,477, 5,264,350).

Bacillus licheniformis production hosts have been genetically modified to remove undesired secreted host cell proteins (WO2003093453, WO2003087149), to reduce red pigment formation (WO2004011609), or antibiotics (WO2003087142, WO2016180928) and therefore ease the subsequent downstream purification process of the desired compound of interest, in particular secreted compounds of interest.

The optimization of the Bacillus licheniformis host cell for the production biological compounds is of high relevance, where even slightly improved Bacillus licheniformis production hosts have a significant impact on large scale industrial production systems and product purity.

Hence, the present invention relates to modified Bacillus licheniformis host cells with decreased host cell proteins and higher purity of the compound of interest.

### BRIEF SUMMARY OF THE INVENTION

It has been found in the studies underlying the present invention that a Bacillus licheniformis host cell comprising at least one of
a) a genetic modification causing compared to an unmodified control cell an increased expression of at least one of the genes selected from the group consisting of degQ, degU, degS, degR, and phrG,
b) a genetic modification causing compared to an unmodified control cell an increased autophosphorylation activity of the DegS protein, and
c) a genetic modification causing compared to an unmodified control cell a reduced phosphatase activity of the DegS protein allows for an efficient production of a compound of interest in said host cell with increased purity.

Thus, the present invention is directed to such modified Bacillus licheniformis host cell, in particular a modified Bacillus host cell comprising an expression cassette for the production of a compound of interest, preferably a polypeptide of interest.

Hence, in another embodiment the present invention is directed to a method for producing a compound of interest, preferably a polypeptide of interest, comprising
a) providing a modified Bacillus licheniformis host cell as described herein,
b) cultivating the host cell under conditions which allow for the expression of the compound of interest, and
c) optionally isolating the compound of interest from the cultivation medium.

### BRIEF DESCRIPTION OF THE FIGURES

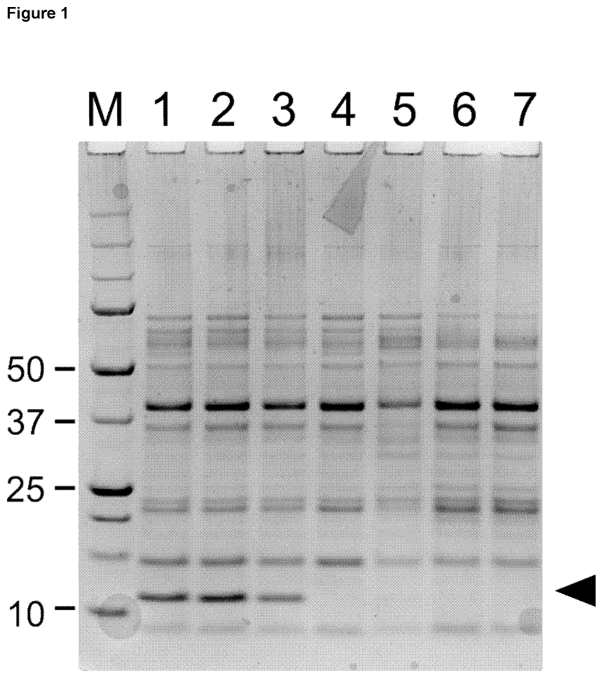

FIG. 1: SDS-PAGE of supernatant from B. licheniformis strains after 72 h of simulated fed-batch cultivation.
1-3=control strain; 4=ForD deletion strain; 5-7=DegQ overexpression strain
Overexpression of DegQ leads to loss of ForD from the extracellular proteome;
M=Precision Plus Protein Standard with indicated size in kDa of selected bands; filled arrowhead-highlights the ForD protein band

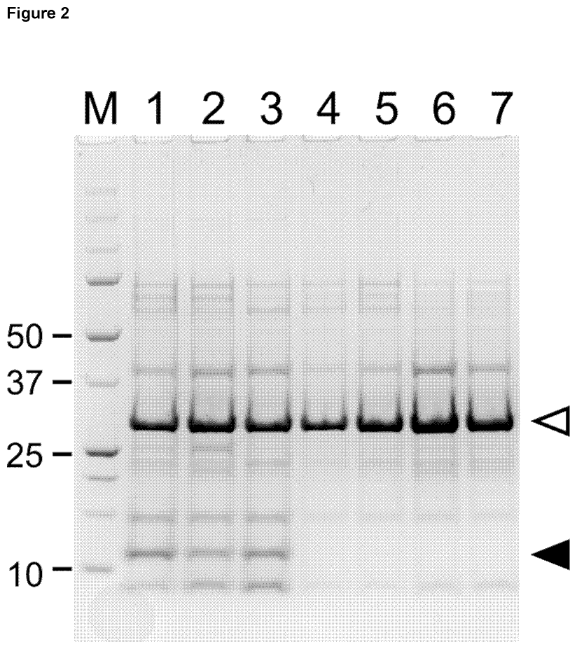

FIG. 2: SDS-PAGE of supernatant from B. licheniformis protease expression strains after 72 h of simulated fed-batch cultivation.
1-3=control strain; 4=ForD deletion strain; 5-7=DegQ overexpression strain;
Overexpression of DegQ leads to loss of ForD from the extracellular proteome; M=Precision
Plus Protein Standard with indicated size in kDa of selected bands; filled arrowhead highlights the ForD protein band; open arrowhead indicates the heterologous protease

### DETAILED DESCRIPTION OF THE INVENTION

It is to be understood that as used in the specification and in the claims, “a” or “an” can mean one or more, depending upon the context in which it is used. Thus, for example, reference to “a cell” can mean that at least one cell can be utilized.

Further, it will be understood that the term “at least one” as used herein means that one or more of the items referred to following the term may be used in accordance with the invention. For example, if the term indicates that at least one feed solution shall be used this may be understood as one feed solution or more than one feed solutions, i.e. two, three, four, five or any other number of feed solutions. Depending on the item the term refers to the skilled person understands as to what upper limit the term may refer, if any.

The term “about” as used herein means that with respect to any number recited after said term an interval accuracy exists within in which a technical effect can be achieved. Accordingly, about as referred to herein, preferably, refers to the precise numerical value or a range around said precise numerical value of ±20%, preferably ±15%, more preferably ±10%, and even more preferably ±5%.

The term “comprising” as used herein shall not be understood in a limiting sense. The term rather indicates that more than the actual items referred to may be present, e.g., if it refers to a method comprising certain steps, the presence of further steps shall not be excluded. However, the term “comprising” also encompasses embodiments where only the items referred to are present, i.e. it has a limiting meaning in the sense of “consisting of”.

The terms “polynucleotide”, “nucleic acid sequence”, “nucleotide sequence”, “nucleic acid”, “nucleic acid molecule” are used interchangeably herein and refer to nucleotides, typically deoxyribonucleotides, in a polymeric unbranched form of any length. The terms “polypeptide” and “protein” are used interchangeably herein and refer to amino acids in a polymeric form of any length, linked together by peptide bonds.

The terms “coding for” and “encoding” are used interchangeably herein. Typically, the terms refer to the property of specific sequences of nucleotides in a polynucleotide, such as a gene, a cDNA, or an mRNA, to serve as templates for synthesis of other macromolecules such as a defined sequence of amino acids. Thus, a gene codes for a protein, if transcription and translation of mRNA corresponding to that gene produces the protein in a cell or other biological system. For the purpose of the present invention, the term “modified”, “genetically modified”, or “genetic modification” (also called herein “recombinant” or “transgenic”) with regard to a cell or an organism means that the cell or organism contains a heterologous polynucleotide which is either obtained from a different organism or generated by man by gene technology. Hence, a modified cell is a non-native cell.

The term “native” (or wildtype or endogenous) cell or organism and “native” (or wildtype or endogenous) polynucleotide or polypeptide refers to the cell or organism as found in nature and to the polynucleotide or polypeptide in question as found in a cell in its natural form and genetic environment, respectively (i.e., without there being any human intervention).

The term “altered protein” as used herein refers to a protein that has been amended by man by gene technology and can be encoded by a modified endogenous gene or by an exogenous gene (also referred to as heterologous to the host cell), e.g., an exogenous gene encoding said protein inserted into a host cell, preferably, along with a deleted or inactivated endogenous gene encoding said protein. Hence, an altered protein is a non-native protein.

The term “nonsense mutation” is a point mutation that leads to a stop codon within the coding region of a protein-encoding sequence.

The term “missense mutation” is a point mutation that leads to another amino acid at the respective amino acid position.

The term “inactivating a gene” means that the expression of the gene has been reduced as compared to expression in a control cell. Preferably, expression of the gene in the bacterial host cell of the present invention has been reduced by at least 40% such as at least 50%, at least 60%, at least 70%, at least 80%, or at least 90% as compared to the corresponding expression in the control cell. More preferably, said expression has been reduced by at least 95%. Most preferably, it has been reduced by 100%, i.e. has been eliminated completely.

A “control cell” as referred to herein is a control cell of the same species which does not carry the respective modification, preferably which differs from the host cell only in that it does not carry the respective modification. Thus, the control cell is an unmodified cell, such as a wild-type cell, i.e. an unmodified wild-type cell, preferably a Bacillus licheniformis cell, which does not carry the respective modification. Preferably, the control cell is a Bacillus licheniformis cell, which differs from the host cell only in that it does not carry the respective modification.

The inactivation of a gene as referred to herein may be achieved by any method deemed appropriate. In an embodiment, the gene has been inactivated by mutation, i.e. by mutating the gene. Preferably, said mutation is a deletion, preferably, said gene has been deleted.

As used herein, the “deletion of a gene” refers to the deletion of the entire coding sequence, deletion of part of the coding sequence, or deletion of the coding sequence including flanking regions with the end result being that the deleted gene is effectively non-functional. In simple terms, a “deletion” is defined as a change in either nucleotide or amino acid sequence in which one or more nucleotides or amino acid residues, respectively, have been removed (i.e., are absent). Thus, a deletion strain has fewer nucleotides or amino acids than the respective wild-type organism.

The term “inactivating a protein” means that the protein is altered in its amino acid sequence in a way that the function of the protein in the cell has been reduced as compared to the non-altered protein. Preferably, the function of the protein in the bacterial host cell of the present invention has been reduced by at least 10%, such as at least 40%, as at least 50%, at least 60%, at least 70%, at least 80%, or at least 90% as compared to the corresponding function of the non-altered protein. More preferably, said function has been reduced by at least 95%. Most preferably, the function has been reduced by 100%, i.e. the protein in completely non-functional. A variant of a parent protein may have an amino acid sequence which has a certain percent identity to the amino acid sequence of the parent sequence. Thus, a variant of a parent polypeptide may comprise an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but below 100% identical to an amino acid sequence of the parent polypeptide. Variants may be, thus, defined by their sequence identity when compared to a parent polypeptide. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

After aligning the two sequences, in a second step, an identity value shall be determined from the alignment. Therefore, according to the present invention the following calculation of percent-identity applies:

%-identity=(identical residues/length of the alignment region which is showing the respective sequence of this invention over its complete length)*100.

Thus, sequence identity in relation to comparison of two amino acid sequences according to this embodiment is calculated by dividing the number of identical residues by the length of the alignment region which is showing the respective sequence of this invention over its complete length. This value is multiplied with 100 to give “%-identity”.

For calculating the percent identity of two DNA sequences the same applies as for the calculation of percent identity of two amino acid sequences with some specifications. For DNA sequences encoding for a protein the pairwise alignment shall be made over the complete length of the coding region from start to stop codon excluding introns. For non-protein-coding DNA sequences the pairwise alignment shall be made over the complete length of the sequence of this invention, so the complete sequence of this invention is compared to another sequence, or regions out of another sequence. Moreover, the preferred alignment program implementing the Needleman and Wunsch algorithm (J. Mol. Biol. (1979) 48, p. 443-453) is “NEEDLE” (The European Molecular Biology Open Software Suite (EMBOSS)) with the programs default parameters (gapopen=10.0, gapextend=0.5 and matrix=EDNAFULL).

In one embodiment, a variant polypeptide comprises 1-30, 1-20, 1-10, or 1-5 amino acid substitutions, preferably, such substitutions are not pertaining to the functional domains of an enzyme. Variants may be defined by their sequence similarity when compared to a parent polypeptide. Sequence similarity usually is provided as “% sequence similarity” or “%-similarity”. For calculating sequence similarity in a first step a sequence alignment has to be generated as described above. In a second step, the percent-similarity has to be calculated, whereas percent sequence similarity takes into account that defined sets of amino acids share similar properties, e.g., by their size, by their hydrophobicity, by their charge, or by other characteristics. Herein, the exchange of one amino acid with a similar amino acid is referred to as “conservative mutation”. Polypeptide variants comprising conservative mutations appear to have a minimal effect on protein folding resulting in certain polypeptide, preferably enzyme, properties being substantially maintained when compared to the polypeptide properties of the parent polypeptide.

For determination of %-similarity according to this invention the following applies, which is also in accordance with the BLOSUM62 matrix (matrix (Henikoff, J. G.; Proc. Natl. Acad. Sci. USA 89, 10915-10919 (1992)), which is one of the most used amino acids substitution matrix for database searching and sequence alignments. An amino acid exchange is defined as similar if the value of the BLOSUM62 substitution matrix for the pair of letters is positive.
Amino acid A is similar to amino acid S
Amino acid D is similar to amino acids E; N
Amino acid E is similar to amino acids D; K; Q
Amino acid F is similar to amino acids W; Y
Amino acid H is similar to amino acids N; Y
Amino acid I is similar to amino acids L; M; V
Amino acid K is similar to amino acids E; Q; R
Amino acid L is similar to amino acids I; M; V
Amino acid M is similar to amino acids I; L; V
Amino acid N is similar to amino acids D; H; S
Amino acid Q is similar to amino acids E; K; R
Amino acid R is similar to amino acids K; Q
Amino acid S is similar to amino acids A; N; T
Amino acid T is similar to amino acid S
Amino acid V is similar to amino acids I; L; M
Amino acid W is similar to amino acids F; Y
Amino acid Y is similar to amino acids F; H; W.

Conservative amino acid substitutions may occur over the full length of the sequence of a polypeptide sequence of a functional protein such as an enzyme. In one embodiment, such mutations are not pertaining to the functional domains of an enzyme. In another embodiment conservative mutations are not pertaining to the catalytic centers of an enzyme.

Therefore, according to the present invention the following calculation of percent-similarity applies:

%-similarity=[(identical residues+similar residues)/length of the alignment region which is showing the respective sequence of this invention over its complete length]*100.

Thus, sequence similarity in relation to comparison of two amino acid sequences herein is calculated by dividing the number of identical residues plus the number of similar residues by the length of the alignment region which is showing the respective sequence of this invention over its complete length. This value is multiplied by 100 to give “%-similarity”.

Especially, variant polypeptide comprising conservative mutations which are at least m percent similar to the respective parent sequences with m being an integer between 50 and 100, preferably 50, 55, 60, 65, 70, 75, 80, 85, 90, 91, 92, 93, 94, 95, 96, 97, 98 or 99 compared to the full length polypeptide sequence, are expected to have essentially unchanged polypeptide properties. In another embodiment conservative mutations are not pertaining to the catalytic centers of an enzyme. In one embodiment, a variant polypeptide comprises 1-30, 1-20, 1-10, or 1-5 conservative amino acid substitutions, preferably, such substitutions are not pertaining to the functional domains of an enzyme.

Likewise, the exchange of one amino acid with a non-similar amino acid is referred to as “non-conservative mutation”. Enzyme variants comprising non-conservative mutations appear to have an effect on protein folding resulting in certain enzyme properties being different when compared to the enzyme properties of the parent enzyme. Hence, an amino acid exchange is defined as non-conservative if the value of the BLOSUM62 substitution matrix for the pair of letters is negative.
Amino acid A is non-similar to amino acids D, E, F, H, I, K, L, M, N, P, Q, R, W, Y
Amino acid C is non-similar to amino acids D, E, F, G, H, I, K, L, M, N, P, Q, R, S, T, V, W, Y
Amino acid D is non-similar to amino acids A, C, F, G, H, I, K, L, M, P, R, T, V, W, Y
Amino acid E is non-similar to amino acids A, C, F, G, I, L, M, P, T, V, W, Y
Amino acid F is non-similar to amino acids A, C, D, E, G, H, K, N, P, Q, R, S, T, V
Amino acid G is non-similar to amino acids C, D, E, F, H, I, K, L, M, P, Q, R, T, V, W, Y
Amino acid H is non-similar to amino acids A, C, D, F, G, I, K, L, M, P, S, T, V, W
Amino acid I is non-similar to amino acids A, C, D, E, G, H, K, N, P, Q, R, S, T, W, Y
Amino acid K is non-similar to amino acids A, C, D, F, G, H, I, L, M, P, T, V, W, Y
Amino acid L is non-similar to amino acids A, C, D, E, G, H, K, N, P, Q, R, S, T, W, Y
Amino acid M is non-similar to amino acids A, C, D, E, G, H, K, N, P, R, S, T, W, Y
Amino acid N is non-similar to amino acids A, C, F, I, L, M, P, V, W, Y
Amino acid P is non-similar to amino acids A, C, D, E, F, G, H, I, K, L, M, N, Q, R, S, T, V, W, Y
Amino acid Q is non-similar to amino acids A, C, F, G, I, L, P, T, V, W, Y
Amino acid R is non-similar to amino acids A, C, D, F, G, I, L, M, P, S, T, V, W, Y
Amino acid S is non-similar to amino acids C, F, H, I, L, M, P, R, V, W, Y
Amino acid T is non-similar to amino acids C, D, E, F, G, H, I, K, L, M, P, Q, R, W, Y
Amino acid V is non-similar to amino acids C, D, E, F, G, H, K, N, P, Q, R, S, W, Y
Amino acid W is non-similar to amino acids A, C, D, E, G, H, I, K, L, M, N, P, Q, R, S, T, V
Amino acid Y is non-similar to amino acids A, C, D, E, G, I, K, L, M, N, P, Q, R, S, T, V

### The Host Cell

The present invention refers to a modified Bacillus licheniformis host cell preferably comprising an increased level of phosphorylated DegU protein compared to an unmodified control cell. “Phosphorylated DegU” is herein also referred to as DegU-P. Preferably, the modified Bacillus licheniformis host cell does not comprise a DegU32 mutation. The term “DegU32 mutation” refers herein to a genetic modification leading to the amino acid substitution H12L in the Bacillus licheniformis DegU protein.

The amount of DegU-P can be preferably increased by mutations in the DegS/DegU two-component regulatory system. In an embodiment, the amount of DegU-P can be increased by a genetic modification causing increased expression of the degU (or a variant thereof), degS (or a variant thereof), degQ (or a variant thereof), degR (or a variant thereof), or phrG (or a variant thereof) gene.

In particular, the present invention relates to a to a modified Bacillus licheniformis host cell comprising at least one of
a. a genetic modification in the Bacillus licheniformis host cell causing compared to an unmodified control cell an increased expression of at least one of the genes selected from the group consisting of degQ, degU, degS, degR, and phrG,
b. a genetic modification in the Bacillus licheniformis host cell causing compared to an unmodified control cell an increased autophosphorylation activity of the DegS protein, and
c. a genetic modification in the Bacillus licheniformis host cell causing compared to an unmodified control cell a reduced phosphatase activity of the DegS protein.

Preferably, the present invention relates to modified Bacillus licheniformis host cell comprising a genetic modification in the Bacillus licheniformis host cell causing compared to an unmodified control cell an increased expression of the degQ gene.

Preferably, the present invention relates to a modified Bacillus licheniformis host cell comprising a genetic modification in the Bacillus licheniformis host cell causing compared to an unmodified control cell an increased expression of the degQ gene and a genetic modification in the Bacillus licheniformis host cell causing compared to an unmodified control cell an increased autophosphorylation activity of the DegS protein, or a genetic modification in the Bacillus licheniformis host cell causing compared to an unmodified control cell a reduced phosphatase activity of the DegS protein.

Preferably, the present invention relates to a to a modified Bacillus licheniformis host cell comprising an increased level of phosphorylated DegU protein compared to an unmodified control cell, wherein the increased level of phosphorylated DegU protein is caused by at least one of
a. a genetic modification in the Bacillus licheniformis host cell causing compared to an unmodified control cell an increased expression of at least one of the genes selected from the group consisting of degQ, degU, degS, degR, and phrG,
b. a genetic modification in the Bacillus licheniformis host cell causing compared to an unmodified control cell an increased autophosphorylation activity of the DegS protein, and
c. a genetic modification in the Bacillus licheniformis host cell causing compared to an unmodified control cell a reduced phosphatase activity of the DegS protein.

Preferably, the present invention relates to a to a modified Bacillus licheniformis host cell comprising an increased level of phosphorylated DegU protein compared to an unmodified control cell, wherein the increased level of phosphorylated DegU protein is caused by a genetic modification in the Bacillus licheniformis host cell causing compared to an unmodified control cell an increased expression the degQ gene.

Preferably, the present invention relates to a to a modified Bacillus licheniformis host cell comprising an increased level of phosphorylated DegU protein compared to an unmodified control cell, wherein the increased level of phosphorylated DegU protein is caused by a genetic modification in the Bacillus licheniformis host cell causing compared to an unmodified control cell an increased expression the degQ gene and a genetic modification in the Bacillus licheniformis host cell causing compared to an unmodified control cell an increased autophosphorylation activity of the DegS protein, or a genetic modification in the Bacillus licheniformis host cell causing compared to an unmodified control cell a reduced phosphatase activity of the DegS protein.

In one embodiment, the Bacillus licheniformis host cell comprises a genetic modification causing compared to an unmodified control cell an increased expression of at least one of the genes selected from the group consisting of degQ, degU, degS, degR, and phrG gene. Preferably, the Bacillus licheniformis host cell comprises a genetic modification causing compared to an unmodified control cell an increased expression of the degQ gene.

Methods for increasing expression of genes or gene products are well documented in the art and include, for example, overexpression driven by appropriate promoters. Appropriate promoters for expressing nucleotide sequences in a host cell are well-known in the art and described elsewhere herein in more detail. Preferably, the promoter sequence for overexpressing any of the degQ, degU, degS, degR, and phrG gene, preferably the degQ gene, comprises a nucleotide sequence having at least 80%, at least 90%, or 100% to nucleotides 19-94 of SEQ ID NO: 3. In some embodiments, the increased expression is achieved by introducing and expressing the gene into the host cell (under control of a suitable promoter). For example, increased expression of degQ can achieved by introducing the degQ gene into the host cell and expressing said degQ gene. Thus, an additional copy of the degQ can be introduced into the host cell. Furthermore, increased expression of a nucleic acid may be achieved by introducing isolated nucleic acids which serve as promoter in an appropriate position (typically upstream) of a gene so as to upregulate expression of a nucleic acid encoding gene. For example, endogenous promoters may be altered in vivo by mutation, deletion, and/or substitution, or isolated promoters may be introduced into a host cell in the proper orientation and distance from the nucleic acid encoding gene so as to increase the expression of the gene.

The terms “increased” and “enhanced” are used interchangeably herein and shall mean with respect to the expression of a gene or protein of interest an increase of transcription and translation of the gene, preferably of at least 10%, at least 20%, at least 25%, at least 30%, at least 40%, at least 50%, at least 60%, at least 70%, at least 80%, at least 90%, or 100%. Preferably, the increase is with respect to a control cell.

The host cell of the present invention may be any Bacillus licheniformis host cell. For example, the host cell may be a host cell of the Bacillus licheniformis strain ATCC14580 (which is the same as DSM13, see Veith et al. “The complete genome sequence of Bacillus licheniformis DSM13, an organism with great industrial potential.” J. Mol. Microbiol. Biotechnol. (2004) 7:204-211).

The increased expression of the at least one of the genes described herein, e.g., degQ, degU, degS, degR, or phrG gene, can be achieved by changing the control sequences of the genes described herein that lead to an increased expression of the encoded protein. Alternatively or in combination thereto, the increased expression of the at least one of the genes described herein can be achieved by increasing the copy number of one or more of the genes described herein. The additional copy of the one or more genes described herein can encode for the native protein or a variant thereof. Thus, the increased expression of the one or more genes described herein can be achieved by overexpressing a native or non-native protein. The one or more additional gene coding for the one or more protein described herein can be integrated into the genome, i.e., chromosome, of the host cell or can be present in an extrachromosomal genetic element, e.g., a plasmid DNA.

In one embodiment, the increased expression of the DegQ, DegU, DegS, DegR, or PhrG protein in the Bacillus licheniformis cell can be achieved by increasing the expression of a DegQ, DegU, DegS, DegR, or PhrG protein from the same Bacillus species, i.e., Bacillus licheniformis. In another embodiment, the increased expression of the DegQ, DegU, DegS, DegR, or PhrG protein in the Bacillus licheniformis cell can be achieved by increasing the expression of a DegQ, DegU, DegS, DegR, or PhrG protein from a different Bacillus species, e.g., a Bacillus species closely related to Bacillus licheniformis, e.g., Bacillus subtilis, Bacillus lentus, or Bacillus pumilus, preferably, from Bacillus subtilis. In yet another embodiment, the increased expression of the DegU, DegS, DegQ, DegR, or PhrG protein in the Bacillus licheniformis cell can be achieved by increasing the expression of a functional variant of a DegQ, DegU, DegS, DegR, or PhrG protein from the a Bacillus species, preferably a variant of the DegQ, DegU, DegS, DegR, or PhrG protein from Bacillus licheniformis.

In one embodiment, the Bacillus licheniformis host cell comprises a genetic modification causing compared to an unmodified control cell an increased expression of the degQ gene.

The degQ gene described herein encodes the DegQ protein. DegQ is a degradation enzyme regulation protein which stimulates the phosphotransfer from phospho-DegS to DegU. Increased expression of the degQ gene results in an improved production of a compound of interest.

In an embodiment, the DegQ protein is from Bacillus licheniformis (or is a variant thereof). Said protein has a sequence as shown in SEQ ID NO: 37 (and is encoded by a polynucleotide comprising a sequence as shown in SEQ ID NO: 35). Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 37.

In an embodiment, the DegQ protein is from Bacillus subtilis (or is a variant thereof). Said protein has a sequence as shown in SEQ ID NO: 38 (and is encoded by a polynucleotide comprising a sequence as shown in SEQ ID NO: 36). Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 38.

In an embodiment, the DegQ protein is from Bacillus velezensis (or is a variant thereof). Said protein has a sequence as shown in SEQ ID NO: 51 (and is encoded by a polynucleotide comprising a sequence as shown in SEQ ID NO: 48). Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 51.

In an embodiment, the DegQ protein is from Bacillus amyloliquefaciens (or is a variant thereof). Said protein has a sequence as shown in SEQ ID NO: 52 (and is encoded by a polynucleotide comprising a sequence as shown in SEQ ID NO: 49). Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 52.

In an embodiment, the DegQ protein is from Bacillus pumilus (or is a variant thereof). Said protein has a sequence as shown in SEQ ID NO: 50 (and is encoded by a polynucleotide comprising a sequence as shown in SEQ ID NO: 47). Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 50.

Preferably, the DegQ protein is from Bacillus licheniformis, preferably has a sequence as shown in SEQ ID NO: 37 or a variant thereof.

In a preferred embodiment, the Bacillus licheniformis host cell comprises a genetic modification causing compared to an unmodified control cell an increased expression of the degQ gene by integration of an additional copy of the degQ gene into the Bacillus licheniformis host cell. Preferably, the Bacillus licheniformis host cell comprises a genetic modification causing compared to an unmodified control cell an increased expression of the degQ gene by introducing into the host cell an expression construct comprising a gene coding for the DegQ protein, preferably comprising a nucleotide sequence having at least 80%, at least 90%, or 100% to nucleotides 11-151 of SEQ ID NO: 4, operably linked to promoter sequence, preferably comprising a nucleotide sequence having at least 80%, at least 90%, or 100% to nucleotides 19-94 of SEQ ID NO: 3. Preferably, the expression construct is integrated as an additional copy of the degQ gene into the chromosome of the Bacillus licheniformis host cell. In a particularly preferred embodiment, the Bacillus licheniformis host cell comprises a genetic modification causing compared to an unmodified control cell an increased expression of the degQ gene by introducing into the host cell in addition to the endogenous degQ gene an expression construct comprising gene coding for the DegQ protein comprising a nucleotide sequence having nucleotides 11-151 of SEQ ID NO: 4, operably linked to promoter sequence comprising nucleotides 19-94 of SEQ ID NO: 3.

In one embodiment, the Bacillus licheniformis host cell comprises a genetic modification causing compared to an unmodified control cell an increased expression of the degU gene. The degU gene described herein encodes the transcriptional regulatory protein DegU. The transcriptional regulatory protein DegU is a member of the two-component regulatory system DegS/DegU which plays an important role in the transition growth phase. Increased expression of the degU gene results in an improved production of a compound of interest.

In an embodiment, the DegU protein is from Bacillus licheniformis (or is a variant thereof). Said protein has a sequence as shown in SEQ ID NO: 54 (and is encoded by a polynucleotide comprising a sequence as shown in SEQ ID NO: 53). Typically, the variant comprises an amino acid sequence which is at least 40%, 50%, 60%, 70%, 80%, 85%, 90%, 91%, 92%, 93%, 94%, 95%, 96%, 97%, 98%, or 99%, but less than 100% identical to SEQ ID NO: 54.

In an embodiment, the DegU protein is from Bacillus subtilis (or is a variant thereof). Said protein has a sequence as shown in SEQ ID NO: 56 (and is encoded by a polynucleotide comprising a sequence as shown in SEQ ID NO: 55). The term “variant” is defined elsewhere herein. Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 56.

The term “Phosphorylated DegU” as used herein preferably refers to the DegU protein which is phosphorylated at a position corresponding to position 56 of SEQ ID NO: 54 or to position 56 of SEQ ID NO: 56.

Preferably, the DegU protein is from Bacillus licheniformis, preferably has a sequence as shown in SEQ ID NO: 54 or a variant thereof.

In one embodiment, the Bacillus licheniformis host cell comprises a genetic modification causing compared to an unmodified control cell an increased expression of the degS gene. The degS gene described herein encodes the DegS protein. DegS is a signal transduction histidine-protein kinase/phosphatase. The kinase plays an important role in the transition growth phase and is involved in the control of expression of different cellular functions. The protein acts as both a protein kinase that undergoes autophosphorylation and transfers the phosphate to DegU. Increased expression of the degS gene results in an improved production of a compound of interest.

In an embodiment, the DegS protein is from Bacillus licheniformis (or is a variant thereof). Said protein has a sequence as shown in SEQ ID NO: 41 (and is encoded by a polynucleotide comprising a sequence as shown in SEQ ID NO: 39). Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 41.

In an embodiment, the DegS protein is from Bacillus subtilis (or is a variant thereof). Said protein has a sequence as shown in SEQ ID NO: 42 (and is encoded by a polynucleotide comprising a sequence as shown in SEQ ID NO: 40). Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% but less than 100% identical to SEQ ID NO: 42.

Preferably, the DegQ protein is from Bacillus licheniformis, preferably has a sequence as shown in SEQ ID NO: 41 or a variant thereof.

In one embodiment, the Bacillus licheniformis host cell comprises a genetic modification causing compared to an unmodified control cell an increased expression of the degR gene.

DegR is regulatory protein which stabilizes the phosphorylated form of DegU. Typically, the stabilization contributes to increased levels of phosphorylated DegU and results in enhanced production of exoenzymes e.g. alkaline protease, and neutral protease. Increased expression of the degR gene results in an improved production of a compound of interest.

In an embodiment, the DegR protein is from Bacillus licheniformis (or is a variant thereof). Said protein has a sequence as shown in SEQ ID NO: 58 (and is encoded by a polynucleotide comprising a sequence as shown in SEQ ID NO: 57). Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 58.

In an embodiment, the DegR protein is from Bacillus subtilis (or is a variant thereof). Said protein has a sequence as shown in SEQ ID NO: 60 (and is encoded by a polynucleotide comprising a sequence as shown in SEQ ID NO: 59). Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 60.

Preferably, the DegR protein is from Bacillus licheniformis, preferably has a sequence as shown in SEQ ID NO: 58 or a variant thereof.

As set forth above, the mutation which increases the amount of phosphorylated DegU may be also a mutation causing increased expression of phrG. Thus, in one embodiment, the Bacillus licheniformis host cell comprises a genetic modification causing compared to an unmodified control cell an increased expression of the phrG gene.

The phrG gene described herein encodes the PhrG polypeptide. The response regulator aspartate phosphatase RapG controls DegU-P activity by interaction with the DNA binding domain of DegU-P thereby preventing the degU-P from binding to its target promoter which is countered by binding of PhrG to RapG Increased expression of the phrG gene results in an improved production of a compound of interest.

In an embodiment, the PhrG protein is from Bacillus licheniformis (or is a variant thereof). Said protein has a sequence as shown in SEQ ID NO: 62 (and is encoded by a polynucleotide comprising a sequence as shown in SEQ ID NO: 61). Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 62.

In an embodiment, the PhrG protein is from Bacillus subtilis (or is a variant thereof). Said protein has a sequence as shown in SEQ ID NO: 64 (and is encoded by a polynucleotide comprising a sequence as shown in SEQ ID NO: 63). Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% but less than 100% identical to SEQ ID NO: 64.

Preferably, the PhrG protein is from Bacillus licheniformis, preferably has a sequence as shown in SEQ ID NO: 62 or a variant thereof.

As set forth above, the mutation which increases the amount of phosphorylated DegU may be also a modification increasing autophosphorylation activity of the DegS protein, such as a DegS-X76D mutation, preferably DegS-S76D mutation. The DegS protein has been defined above. The DegS-X76D mutant comprises a X76D substitution, i.e. a substitution at position 76 according to the numbering of SEQ ID NO: 41 to Aspartic Acid, preferably the amino acid Serine at position 76 is replaced with Aspartic Acid at amino acid position 76 of the DegS protein according to the numbering of SEQ ID NO: 41. In an embodiment, the DegS-X76D mutant polypeptide comprises an amino acid sequence as shown in SEQ ID NO: 43 or 44, preferably SEQ ID NO: 43, or is a variant thereof. It is to be understood that the variant comprises the X76D, preferably S76D, substitution. Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 43 or 44, preferably SEQ ID NO: 43, wherein the variant comprises the X76D substitution, preferably the S76D substitution. Preferably, the mutant polypeptide is expressed in the modified host cell. This can be achieved by introducing and expressing encoding the mutant polypeptide (or variant thereof) in the host cell. Preferably, the polynucleotide is operably linked to a suitable promoter.

As set forth above, the mutation which increases the amount of phosphorylated DegU may be also a genetic modification reducing phosphatase activity of the DegS protein, such as a degS200 mutation. The DegS protein has been defined above. The degS200 mutation, also referred to as DegS-X218E mutant, comprises a X218E substitution, preferably G218E, i.e. a substitution at position 218 according to the numbering of SEQ ID NO: 41 to Glutamic Acid, preferably the amino acid Glycine at position 218 is replaced with Glutamic Acid at amino acid position 218 of the DegS protein according to the numbering of SEQ ID NO: 41. In an embodiment, the DegS-X218E mutant polypeptide comprises an amino acid sequence as shown in SEQ ID NO: 45 or 46, preferably SEQ ID NO: 45, or is a variant thereof. It is to be understood that the variant comprises the X218E substitution, preferably the G218E substitution. However, it is understood within the scope of the invention that an amino acid exchange X218D, preferably G218D, would lead to the same effect. Typically, the variant comprises an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99%, but less than 100% identical to SEQ ID NO: 45 or 46, wherein the variant comprises the X218E, preferably, the G218E or G218D substitution. Preferably, the mutant polypeptide is expressed in the modified host cell. This can be achieved by introducing and expressing encoding the mutant polypeptide (or variant thereof) in the host cell. Preferably, the polynucleotide is operably linked to a suitable promoter.

In an embodiment, the host cell comprises two genetic modifications which increase the amount of phosphorylated DegU. For example, the host cell comprises a genetic modification causing increased expression of degQ and a degU32 mutation.

In an embodiment, the host cell comprises two genetic modifications which increase the amount of phosphorylated DegU. For example, the host cell comprises a genetic modification causing increased expression of at least one the degQ, degU, degS, degR, and phrG gene and a DegS-X76D mutation.

In another embodiment, the host cell comprises a genetic modification causing increased expression of the at least one the degQ, degU, degS, degR, and phrG gene and a DegS-X218E mutation.

Preferably, the host cell comprises two genetic modifications which increase the amount of phosphorylated DegU. For example, the host cell comprises a genetic modification causing increased expression of at least one the degQ gene and a DegS-X76D mutation.

Preferably, the host cell comprises a genetic modification causing increased expression of the at least one the degQ gene and a DegS-X218E mutation.

Thus, in one embodiment the present invention is directed to a method of producing the modified Bacillus licheniformis host cell described herein. Preferably, the modified Bacillus licheniformis host cell can be obtained by a method comprising the steps of
a) providing a Bacillus licheniformis cell, and
b) modifying the Bacillus cell provided under a) by introducing into the Bacillus licheniformis cell at least one of
   a. a genetic modification in the Bacillus licheniformis host cell causing compared to an unmodified control cell an increased expression of at least one of the genes selected from the group consisting of degQ, degU, degS, degR, and phrG,
   b. a genetic modification in the Bacillus licheniformis host cell causing compared to an unmodified control cell an increased autophosphorylation activity of the DegS protein, and
   c. a genetic modification in the Bacillus licheniformis host cell causing compared to an unmodified control cell a reduced phosphatase activity of the DegS protein.

Preferably, the modified Bacillus licheniformis host cell comprises no modification in the endogenous forD gene.

In another embodiment, the modified Bacillus licheniformis host cell can be obtained by a method comprising the steps of
a) providing a Bacillus licheniformis cell,
b) modifying the Bacillus cell provided under a) by introducing a nucleic acid construct comprising at least one of the genes selected from the group consisting of degQ, degU, degS, degR, and phrG as described herein, preferably, derived from the native Bacillus licheniformis, or selected from the group of a gene coding for a DegS protein comprising an increased autophosphorylation activity and a gene coding for a DegS protein comprising a reduced phosphatase activity, preferably under the control of a suitable promoter sequence, into the Bacillus cell and thereby obtaining the modified Bacillus host cell, and
c) optionally inactivating, preferably deleting, at least one of the endogenous degQ, degU, degS, degR, and phrG gene, preferably maintaining the endogenous degQ, degU, degS, degR, and phrG gene.

In another embodiment, the modified Bacillus licheniformis host cell can be obtained by a method comprising the steps of
a) providing a Bacillus licheniformis cell, and
b) modifying the Bacillus cell provided under a) by introducing a nucleic acid construct comprising at least one of the genes selected from the group consisting of degQ, degU, degS, degR, and phrG as described herein, preferably, derived from the native Bacillus licheniformis, preferably under the control of a suitable promoter sequence, into the Bacillus cell and thereby obtaining the modified Bacillus host cell, and
c) preferably maintaining the endogenous degQ, degU, degS, degR, and phrG gene.

In another embodiment, the modified Bacillus licheniformis host cell can be obtained by a method comprising the steps of
a) providing a Bacillus licheniformis cell,
b) modifying the Bacillus cell provided under a) by introducing a nucleic acid construct comprising at least one of the genes selected from the group consisting of a gene coding for a DegS protein comprising an increased autophosphorylation activity as described herein (preferably the DegS-S76D mutation) and a gene coding for a DegS protein comprising a reduced phosphatase activity as described herein (preferably the DegS-200 mutation), preferably under the control of a suitable promoter sequence, into the Bacillus cell and thereby obtaining the modified Bacillus host cell, and
c) optionally inactivating, preferably deleting, at least one of the endogenous degS gene.

In another embodiment, the modified Bacillus licheniformis host cell can be obtained by a method comprising the steps of
a) providing a Bacillus licheniformis cell,
b) modifying the Bacillus cell provided under a) by modifying the endogenous gene coding for the DegS protein to a variant of the DegS protein comprising an increased autophosphorylation activity as described herein (preferably by introducing the DegS-S76D mutation) or by modifying the endogenous gene coding for the DegS protein to a variant of the DegS protein comprising a reduced phosphatase activity as described herein (preferably by introducing the DegS-200 mutation), preferably under the control of a suitable promoter sequence, into the Bacillus cell and thereby obtaining the modified Bacillus host cell, and
c) optionally inactivating, preferably deleting, at least one of the endogenous degS gene.

In another embodiment, the modified Bacillus licheniformis host cell can be obtained by a method comprising the steps of
a) providing a Bacillus licheniformis cell,
b) modifying the Bacillus cell provided under a) by introducing a nucleic acid construct comprising at least one of the genes selected from the group consisting of degQ, degU, degS, degR, and phrG as described herein, preferably, derived from the native Bacillus licheniformis, preferably under the control of a suitable promoter sequence, into the Bacillus cell and by introducing a nucleic acid construct comprising at least one of the genes selected from the group consisting of a gene coding for a DegS protein comprising an increased autophosphorylation activity as described herein (preferably the DegS-S76D mutation) and a gene coding for a DegS protein comprising a reduced phosphatase activity as described herein (preferably the DegS-200 mutation), preferably under the control of a suitable promoter sequence, into the Bacillus cell and thereby obtaining the modified Bacillus host cell, and
c) inactivating, preferably deleting, at least one of the endogenous degS gene.

In another embodiment, the modified Bacillus licheniformis host cell can be obtained by a method comprising the steps of
a) providing a Bacillus licheniformis cell,
b) modifying the Bacillus cell provided under a) by introducing a nucleic acid construct comprising a degQ gene as described herein, preferably, derived from the native Bacillus licheniformis, preferably under the control of a suitable promoter sequence, into the Bacillus cell and by introducing a nucleic acid construct comprising at least one of the genes selected from the group consisting of a gene coding for a DegS protein comprising an increased autophosphorylation activity as described herein (preferably the DegS-S76D mutation) and a gene coding for a DegS protein comprising a reduced phosphatase activity as described herein (preferably the DegS-200 mutation), preferably under the control of a suitable promoter sequence, into the Bacillus cell and thereby obtaining the modified Bacillus host cell, and
c) inactivating, preferably deleting, at least one of the endogenous degS gene.

In another embodiment, the modified Bacillus licheniformis host cell can be obtained by a method comprising the steps of
a) providing a Bacillus licheniformis cell,
b) modifying the Bacillus cell provided under a) by introducing a nucleic acid construct comprising a degQ gene as described herein, preferably, derived from the native Bacillus licheniformis, preferably under the control of a suitable promoter sequence, into the Bacillus cell and by modifying the endogenous gene coding for the DegS protein to a variant of the DegS protein comprising an increased autophosphorylation activity as described herein (preferably by introducing the DegS-S76D mutation) or by modifying the endogenous gene coding for the DegS protein to a variant of the DegS protein comprising a reduced phosphatase activity as described herein (preferably by introducing the DegS-200 mutation), preferably under the control of a suitable promoter sequence, into the Bacillus cell and thereby obtaining the modified Bacillus host cell, and
c) inactivating, preferably deleting, at least one of the endogenous degS gene.

In one embodiment, the Bacillus host cell is used for producing a compound of interest as described elsewhere herein. The compound of interest can be endogenous or heterologous to the host cell. Preferably, the compound of interest is a protein, preferably an enzyme. Preferably the compound of interest is heterologous to the host cell. Preferably, the compound of interest is a protein, preferably an enzyme, heterologous to the host cell.

In one embodiment, the host cell comprises an expression cassette for the production of a compound of interest, preferably a polypeptide of interest. In one embodiment, the polypeptide of interest is an enzyme, such as an enzyme selected from the group consisting of amylase, protease, lipase, phospholipase, mannanase, phytase, xylanase, lactase, phosphatase, glucoamylase, nuclease, galactosidase, endoglucanase and cellulase, preferably a protease and/or an amylase.

Preferably, the modified Bacillus licheniformis host cell described herein comprises reduced expression of the gene coding for Formosin D (ForD, bacteriocin), preferably compared to a Bacillus licheniformis control cell that does not comprise modification described herein. Preferably, the modified Bacillus licheniformis host cell comprises no modification in the endogenous forD gene, i.e., the modified Bacillus licheniformis host cell still comprises an unmodified endogenous forD gene. Preferably, the modified Bacillus licheniformis host cell described herein comprises a reduced expression of the gene coding for the Formosin D protein, preferably compared to a Bacillus licheniformis control cell that does not comprise modification described herein, wherein the Formosin D protein is encoded by a sequence having with increased preference at least 60%, at least 70%, at least 80%, at least 90%, at least 95%, at least 99%, or 100% identity to SEQ ID NO: 33. Preferably, the modified Bacillus licheniformis host cell described herein comprises a reduced expression of the gene coding for the Formosin D protein, preferably compared to a Bacillus licheniformis control cell that does not comprise modification described herein, wherein the Formosin D protein has with increased preference at least 60%, at least 70%, at least 80%, at least 90%, at least 95%, at least 99%, or 100% identity to SEQ ID NO: 34. Preferably, the modified Bacillus licheniformis host cell described herein comprises a reduced expression of the gene coding for the Formosin D protein, preferably compared to a Bacillus licheniformis control cell that does not comprise modification described herein, wherein the Formosin D protein has with increased preference at least 60%, at least 70%, at least 80%, at least 90%, at least 95%, at least 99%, or 100% identity to SEQ ID NO: 34.

Preferably, the modified Bacillus licheniformis host cell described herein produces a compound of interest, preferably a polypeptide of interest, with increased purify, preferably compared to a Bacillus licheniformis control cell that does not comprise modification described herein. Thus, preferably, the modified Bacillus licheniformis host cell described herein comprises an increased purity of the compound of interest, preferably compared to a Bacillus licheniformis control cell that does not comprise modification described herein. Preferably, the modified Bacillus licheniformis host cell described herein produces a compound of interest, preferably a polypeptide of interest, with reduced amount of Formosin D (ForD), preferably compared to a Bacillus licheniformis control cell that does not comprise modification described herein. Preferably, the modified Bacillus licheniformis host cell described herein produces a compound of interest, preferably a polypeptide of interest, with reduced amount of the Formosin D protein, preferably compared to a Bacillus licheniformis control cell that does not comprise modification described herein, wherein the Formosin D protein has with increased preference at least 60%, at least 70%, at least 80%, at least 90%, at least 95%, at least 99%, or 100% identity to SEQ ID NO: 34. Preferably, the modified Bacillus licheniformis host cell described herein comprises an increased production of the compound of interest, preferably compared to a Bacillus licheniformis control cell that does not comprise modification described herein. Preferably, the modified Bacillus licheniformis host cell described herein comprises an increased purity of the compound of interest, preferably with reduced amount of Formosin D, preferably compared to a Bacillus licheniformis control cell that does not comprise modification described herein and comprises an increased production of the compound of interest, preferably compared to a Bacillus licheniformis control cell that does not comprise modification described herein.

Methods for the generation of modified Bacillus cells and altered proteins, e.g., by introduction of foreign nucleic acids, chromosomal gene deletion, substitution, mutation, and inactivation, are known in the art.

The introduction of DNA into a host cell, in one embodiment a Bacillus cell, may, for instance, be effected by protoplast transformation (see, e.g., Chang and Cohen, 1979, Molecular General Genetics 168: 111-115), by using competent cells (see, e.g., Young and Spizizen, 1961, Journal of Bacteriology 81: 823-829, or Dubnau and Davidoff-Abelson, 1971, Journal of Molecular Biology 56: 209-221), by electroporation (see, e.g., Shigekawa and Dower, 1988, Biotechniques 6: 742-751), or by conjugation (see, e.g., Koehler and Thorne, 1987, Journal of Bacteriology 169: 5271-5278). Specific transformation protocols are known in the art for various types of host cells (see, e.g., for E. coli protoplast transformation see Hanahan, 1983, J. Mol. Biol. 166: 557-580). Gene inactivation can be achieved by homologous recombination, i.e. an incoming DNA molecule comprises sequences that are homologous to the 5′ and 3′ flanking sequences of the target sequence on the chromosome of the host cell (e.g. Bacillus) to be inactivated. Subsequently the sequence between said flanking sequences is replaced by the homologous sequences of the incoming DNA molecule in the process of homologous recombination, i.e. the sequence is deleted from the chromosome. Likewise “gene integration”, i.e. a DNA sequence such as a gene expression cassette with or without a selectable marker, can be integrated into the chromosome of the bacterial host cell by homologous recombination. Hence. The DNA sequence to be integrated is flanked by DNA sequences that are homologous to the 5′ and 3′ flanking sequences on the chromosome. It is understood in terms of the invention that gene integration can also combine gene integration and gene deletion in one step, i.e. a DNA sequence on the chromosome is replaced by the incoming DNA sequence for gene integration.

Homologous recombination can be achieved by two different methods known in the art: By two consecutive rounds of homologous recombination (Campbell recombination) with circular plasmid DNA, e.g. based on the well-known temperature sensitive plasmid pE194 (Nahrstedt et al., Strain development in Bacillus licheniformis: construction of biologically contained mutants deficient in sporulation and DNA repair. J Biotechnol. 2005 Sep. 29; 119(3):245-54). The integration of the deletion plasmid containing an incoming DNA molecule comprising sequences that are homologous to the 5′ and 3′ flanking sequences of the target sequence on the chromosome is achieved by a first homologous recombination (Campbell recombination) with the first homologous region under selective conditions for the selectable marker and cultivation at the non-permissive temperature, i.e. that blocks plasmid replication. The second homologous recombination with the second homologous region is achieved by removal of selective pressure and cultivation at the permissive temperature, i.e. plasmid replication takes place, resulting in excision of the plasmid from the chromosome.

Alternatively, a non-replicative ‘suicide’ plasmid can be used forcing the integration by selection on the selectable marker. Only cells that have integrated the plasmid into the genome by homologous recombination are able to grow under the selective conditions. Plasmid removal/excision from the chromosome is achieved with a second homologous recombination which is forced by the activation of a counterselection marker present on the plasmid.

The second method of homologous recombination refers to two homologous recombination events simultaneously taking place, also known as ‘double crossing over’ or ‘double homologous recombination. The incoming DNA sequence is linear and can be obtained by PCR, linearization of plasmid DNA or preparation of chromosomal DNA which inevitable results in fragmented linear DNA. WO0308125 uses linear DNA constructs (either linearized plasmids or PCR fragments) comprising a selectable marker flanked by the 5′ and 3′ homologous regions which are used for genomic integration via double crossing over homologous recombination. It is well understood that next to the selectable marker additional DNA, such as gene expression cassettes, when flanked by said homologous region are integrated into the chromosome of the bacterial host cell.

Homologous recombination requires DNA sequences homologous to the 5′ and 3′ flanking sequences of the target sequence on the chromosome of the host cell of sufficient size, hence should contain a sufficient number of nucleic acid such as 100 to 1,500 base pairs, preferably 400 to 1,500 base pairs, and most preferably 800 to 1,500 base pairs, which have a high degree of identity to the corresponding target sequence to enhance the probability of homologous recombination (Dubnau, 1993, Genetic exchange and homologous recombination. In Bacillus subtilis and Other Gram-positive Bacteria, p. 555-584. Edited by A. I. Sonenshein, J. A. Hoch & R. Losick, Washington DC, American Society for Microbiology; Michel and Ehrlich, 1984, The EMBO Journal, vol. 3, pp. 2879-2884).

Gene inactivation by deletion/insertion/substitution can also be achieved by CRISPR/Cas9 genome editing technologies where the CRISPR cutting properties could be used to disrupt genes in almost any organism's genome with unprecedented ease (Mali P, et al (2013) Science. 339(6121):819-823; Cong L, et al (2013) Science 339(6121)). Recently it became clear that providing a template for repair, e.g. homologous regions, allowed for editing the genome with nearly any desired sequence at nearly any site, transforming CRISPR into a powerful gene editing tool (WO/2014/150624, WO/2014/204728).

CRISPR-based genome editing systems for application in gram positive organisms have been well described such as the Bacillus species based single-plasmid system approach, i.e. comprising the Cas9 endonuclease, the gRNA (e.g. sgRNA or crRNA/tracrRNA), repair homology sequences (donor DNA) on one single E. coli-Bacillus shuttle vector (Altenbuchner, (2016): Applied and environmental microbiology 82 (17), 5421-5427; Zhou, et al. (2019): International journal of biological macromolecules 122, 329-337), or dual plasmid system or with Cas9 endonuclease integrated into the Bacillus genome as described e.g. in WO2020206202 and WO2020206197.

Alternatively to “directed” methods of inactivation it is understood in the scope of the invention that whole-cell mutagenesis by applying mutagenizing conditions such as exposure of the cells to UV radiation, or chemical mutagenizing chemicals such as NTG (N-methyl-N′-nitro-N-nitrosoguanidine), EMS (ethyl-methane-sulfonate), in combination with screening and/-or selection of the desired property, e.g. reduced lipase/esterase activity is a well-known approach to achieve functional inactivation.

Further, a gene may have been inactivated by gene silencing. Gene silencing can be achieved by introducing into said bacterial host cell antisense expression constructs that result in antisense RNAs complementary to the mRNA of the gene, thereby inhibiting expression of said genes. Alternatively, the expression of said gene can be inhibited by blocking transcriptional initiation or transcriptional elongation through the mechanism of CRISPR-inhibition (WO18009520).

### The Method for Producing a Compound of Interest

In another embodiment, the present invention refers to a method for producing a compound of interest, preferably a polypeptide of interest, preferably with increased purity. For producing the polypeptide of interest, the modified Bacillus licheniformis host cell shall comprise at least one polynucleotide encoding one or more polypeptides of interest, wherein said one or more polynucleotides are operably linked to a promoter. Accordingly, the host cell shall comprise an expression cassette for at least one polypeptide of interest.

Thus, the present invention relates to a method for producing a compound of interest, preferably a polypeptide of interest, comprising
a) providing the modified Bacillus licheniformis host cell comprising at least one of
   a. a genetic modification causing compared to an unmodified control cell an increased expression of at least one of the genes selected from the group consisting of degQ, degU, degS, degR, and phrG,
   b. a genetic modification causing compared to an unmodified control cell an increased autophosphorylation activity of the DegS protein, and
   c. a genetic modification causing compared to an unmodified control cell a reduced phosphatase activity of the DegS protein,
b) introducing onto said modified Bacillus host cell an expression cassette for a compound of interest, preferably a polypeptide of interest, preferably a polypeptide that is secreted by the host cell,
c) cultivating the host cell under conditions which allow for the expression of the compound of interest, and
d) optionally isolating the compound of interest from the cultivation medium, preferably whereby the Bacillus licheniformis host cell produced the compound of interest with increased purity compared to an unmodified control cell.

Preferably, the modified Bacillus licheniformis host cell comprises no modification in the endogenous forD gene.

Preferably, the present invention relates to a method for producing a compound of interest, preferably a polypeptide of interest, comprising
a) providing the modified Bacillus licheniformis host cell comprising a genetic modification causing compared to an unmodified control cell an increased expression of at least one of the degQ gene,
b) introducing onto said modified Bacillus host cell an expression cassette for a compound of interest, preferably a polypeptide of interest, preferably a polypeptide that is secreted by the host cell,
c) cultivating the host cell under conditions which allow for the expression of the compound of interest, and
d) optionally isolating the compound of interest from the cultivation medium.

Preferably, the present invention relates to a method for producing a compound of interest, preferably a polypeptide of interest, comprising
a) providing the modified Bacillus licheniformis host cell comprising
   a. a genetic modification causing compared to an unmodified control cell an increased expression of at least one of the degQ gene, and optionally
   b. a genetic modification causing compared to an unmodified control cell an increased autophosphorylation activity of the DegS protein, or
   c. a genetic modification causing compared to an unmodified control cell a reduced phosphatase activity of the DegS protein,
b) introducing onto said modified Bacillus host cell an expression cassette for a compound of interest, preferably a polypeptide of interest, preferably a polypeptide that is secreted by the host cell,
c) cultivating the host cell under conditions which allow for the expression of the compound of interest, and
d) optionally isolating the compound of interest from the cultivation medium.

The present invention further related to a method for increasing the purity of a compound of interest, preferably a polypeptide of interest, produced by a Bacillus licheniformis cell, comprising the steps of
a) providing a modified Bacillus licheniformis host cell comprising at least one of
   a. a genetic modification causing compared to an unmodified control cell an increased expression of at least one of the genes selected from the group consisting of degQ, degU, degS, degR, and phrG,
   b. a genetic modification causing compared to an unmodified control cell an increased autophosphorylation activity of the DegS protein, and
   c. a genetic modification causing compared to an unmodified control cell a reduced phosphatase activity of the DegS protein,
b) cultivating the host cell under conditions which allow for the expression of the compound of interest, and
c) isolating the compound of interest from the cultivation medium.

The present invention further related to a method for increasing the purity of a compound of interest, preferably a polypeptide of interest, produced by a Bacillus licheniformis cell, comprising the steps of
a) providing a modified Bacillus licheniformis host cell comprising a genetic modification causing compared to an unmodified control cell an increased expression of a degQ gene, b) cultivating the host cell under conditions which allow for the expression of the compound of interest, and
c) isolating the compound of interest from the cultivation medium.

The present invention further related to a method for increasing the purity of a compound of interest, preferably a polypeptide of interest, produced by a Bacillus licheniformis cell, comprising the steps of
a) providing a modified Bacillus licheniformis host cell comprising
   a. a genetic modification causing compared to an unmodified control cell an increased expression of a degQ gene, and optionally
   b. a genetic modification causing compared to an unmodified control cell an increased autophosphorylation activity of the DegS protein, or
   c. a genetic modification causing compared to an unmodified control cell a reduced phosphatase activity of the DegS protein,
b) cultivating the host cell under conditions which allow for the expression of the compound of interest, and
c) isolating the compound of interest from the cultivation medium.

The method of the present invention, if applied, allows for increasing the purity of the compound of interest. Preferably, the purity regarding a particular contamination is increased by at least 10%, at least 20%, at least 30%, at least 40%, at least 50%, at least 60%, at least 70%, at least 80%, at least 90%, or 100% as compared to the purity of the compound of interest produced by a control cell.

Preferably, the Bacillus licheniformis host cell comprises a reduced expression of Formosin D compared to an unmodified control cell, preferably, wherein the Formosin D comprises with increasing preference a sequence having at least 80%, at least 90%, at least 95%, at least 99%, or 100% identity to SEQ ID NO: 34. Preferably, the modified Bacillus licheniformis host cell comprises no modification in the endogenous forD gene.

The present invention further related to a method for reducing the Formosin D contamination of a compound of interest, preferably a polypeptide of interest, produced by a Bacillus licheniformis cell, comprising the steps of
a) providing a modified Bacillus licheniformis host cell comprising at least one of
   a. a genetic modification causing compared to an unmodified control cell an increased expression of at least one of the genes selected from the group consisting of degQ, degU, degS, degR, and phrG,
   b. a genetic modification causing compared to an unmodified control cell an increased autophosphorylation activity of the DegS protein, and
   c. a genetic modification causing compared to an unmodified control cell a reduced phosphatase activity of the DegS protein,
b) cultivating the host cell under conditions which allow for the expression of the compound of interest, and
c) isolating the compound of interest from the cultivation medium.

The method of the present invention, if applied, allows for reducing the contamination of the compound of interest with host cell protein, preferably the Formosin D. Preferably, the contamination of the compound of interest by a particular host cell compound, preferably Formosin D, is reduced by at least 10%, at least 20%, at least 30%, at least 40%, at least 50%, at least 60%, at least 70%, at least 80%, at least 90%, or 100% as compared to the contamination of the compound of interest produced by a control cell.

The explanations and definitions given herein above in connection with the modified host cell of the present invention apply mutatis mutandis to the method of the present invention.

The term “cultivating” as used herein refers to keeping alive and/or propagating the modified host cell comprised in a culture at least for a predetermined time. The term encompasses phases of exponential cell growth at the beginning of growth after inoculation as well as phases of stationary growth. The cultivation conditions shall allow for the expression, i.e. the production, of the polypeptide of interest. Such conditions can be chosen by the skilled person without further ado. Exemplary conditions for the cultivation of the modified host cell are described in Example 1 and 2. In an embodiment of the method of the present invention, the cultivation is carried out as fedbatch cultivation.

The method of the present invention, if applied, allows for increasing the expression, i.e. the production, of the at least one compound, preferably polypeptide, of interest. Preferably, expression is increased as compared to the expression in an unmodified control cell. In a preferred embodiment, expression of the at least one polypeptide of interest is increased by at least 10%, 20% or by at least 40%, such as by at least 50%, or at least 80% as compared to the expression in the control cell. For example, expression of the at least one polypeptide of interest may be increased by 20% to 100%, such as by 40% to 60%, as compared to the control cell. Further, it is envisaged that the expression is increased by at least 100%, 150%, 200%. 250% or 300%, such as by 200% to 300%. Typically, the expression can be measured by determining the amount of the compound of interest in the host cell and/or in the cultivation medium.

In one embodiment, the expression cassette for the expression of the compound of interest in the Bacillus host cell is heterologous to the Bacillus host cell. Preferably, the polynucleotide encoding at least one polypeptide of interest is heterologous to the Bacillus host cell. Preferably, in one embodiment, the polynucleotide encoding at least one polypeptide of interest is heterologous to the bacterial host cell. The term “heterologous” (or exogenous or foreign or recombinant or non-native) polypeptide or protein as used throughout the specification is defined herein as a polypeptide or protein that is not native to the host cell. Similarly, the term “heterologous” (or exogenous or foreign or recombinant or non-native) polynucleotide refers to a polynucleotide that is not native to the host cell.

Preferably, the expression construct encoding for the polypeptide of interest also encodes for a signal sequence that guides secretion of the polypeptide of interest out of the cell, preferably into the cultivation medium (fermentation broth). Such signal peptides are well known in the art and are preferably added to the N-terminus of the polypeptide of interest, preferably linked via a cleavage site so that the signal peptide will be cleaved-off the polypeptide of interest prior or during secretion of the polypeptide of interest outside of the host cell.

In an embodiment, the at least one polynucleotide encoding a polypeptide of interest is present on a plasmid. The term “plasmid” refers to an extrachromosomal circular DNA, i.e. a vector that is autonomously replicating in the host cell. Thus, a plasmid is understood as extrachromosomal vector.

In a preferred embodiment, the replication of a plasmid shall be independent of the replication of the chromosome of the bacterial host cell. For autonomous replication, the expression vector may further comprise an origin of replication enabling the vector to replicate autonomously in the host cell in question. Bacterial origins of replication include but are not limited to the origins of replication of plasmids pBR322, pUC19, pSC101, pACYC177, and pACYC184 permitting replication in E. coli (Sambrook, J. and Russell, D. W. Molecular cloning. A laboratory manual, 3rd ed, Cold Spring Harbor Laboratory Press, Cold Spring Harbor, N Y. 2001; Cohen, S. N., Chang, A. C. Y., Boyer, H. W., & Helling, R. B. (1973). Construction of Biologically Functional Bacterial Plasmids In Vitro. Proceedings of the National Academy of Sciences of the United States of America, 70(11), 3240-3244), and pUB110, pC194, pE194, pTB19, pAMß1, and pTA1060 permitting replication in Bacillus (Janniere, L., Bruand, C., and Ehrlich, S. D. (1990). Structurally stable Bacillus subtilis cloning vectors. Gene 87, 53-6; Ehrlich, S. D., Bruand, C., Sozhamannan, S., Dabert, P., Gros, M. F., Janniere, L., and Gruss, A. (1991). Plasmid replication and structural stability in Bacillus subtilis. Res. Microbiol. 142, 869-873), and pE194 (Dempsey, L. A. and Dubnau, D. A. (1989). Localization of the replication origin of plasmid pE194. J. Bacteriol. 171, 2866-2869). The origin of replication may be one having a mutation to make its function temperature-sensitive in the host cell (see, e.g., Ehrlich, 1978, Proceedings of the National Academy of Sciences USA 75:1433-1436).

The copy number of a plasmid is defined as the average number of plasmids per bacterial cell or per chromosome under normal growth conditions. Moreover, there are different types of replication origins that result in different copy numbers in the bacterial host. The plasmid replicon pBS72 (accession number AY102630.1) and the plasmids pTB19 and derivatives pTB51, pTB52 confer low copy number with 6 copies and 1 to 8 copies, respectively, within Bacillus cells whereas plasmids pE194 (accession number V01278.1) and pUB110 (accession number M19465.1)/pBC16 (accession number U32369.1) confer low-medium copy number with 14-20 and medium copy number with 30-50 copies per cell, respectively. Plasmid pE194 was analyzed in more detail (Villafane, et al (1987): J. Bacteriol. 169(10), 4822-4829) and several pE194-cop mutants described having high copy numbers within Bacillus ranging from 85 copies to 202 copies. Moreover, plasmid pE194 is temperature sensitive with stable copy number up to 37° C., however abolished replication above 43° C. In addition, it exists a pE194 variant referred to as pE194ts with two point mutations within the cop-repF region (nt 1235 ad nt 1431) leading to a more drastic temperature sensitivity—stable copy number up to 32° C., however only 1 to 2 copies per cell at 37° C.

In one embodiment, the vectors contain one or more selectable markers that permit easy selection of transformed cells. A selectable marker is a gene encoding a product, which provides for biocide resistance, resistance to heavy metals, prototrophy to auxotrophs, and the like. Bacterial selectable markers include but are not limited to the dal genes from Bacillus subtilis or Bacillus licheniformis, or markers that confer antibiotic resistance such as ampicillin, kanamycin, erythromycin, chloramphenicol or tetracycline resistance. Furthermore, selection may be accomplished by co-transformation, e.g., as described in WO91/09129, where the selectable marker is on a separate vector.

In another embodiment, the at least one polynucleotide encoding a polypeptide of interest is stably integrated into the bacterial chromosome.

### Promoter

The at least one polynucleotide encoding a polypeptide of interest shall be operably linked to a promoter.

The term “operably linked” as used herein refers to a functional linkage between the promoter sequence and the polynucleotide encoding a polypeptide of interest, such that the promoter sequence is able to initiate transcription of the polynucleotide encoding a polypeptide of interest (herein also referred to as gene of interest).

A “promoter” or “promoter sequence” is a nucleotide sequence located upstream of a gene on the same strand as the gene that enables that gene's transcription. A promoter is followed by the transcription start site of the gene. A promoter is recognized by RNA polymerase (together with any required transcription factors), which initiates transcription. A functional fragment or functional variant of a promoter is a nucleotide sequence which is recognizable by RNA polymerase, and capable of initiating transcription.

An “active promoter fragment”, “active promoter variant”, “functional promoter fragment” or “functional promoter variant” describes a fragment or variant of the nucleotide sequences of a promoter, which still has promoter activity.

A promoter can be an “inducer-dependent promoter” or an “inducer-independent promoter” comprising constitutive promoters or promoters which are under the control of other cellular regulating factors.

The person skilled in the art is capable to select suitable promoters for expressing the third alanine racemase and the polypeptide of interest. For example, the polynucleotide encoding the polypeptide of interest is, preferably, operably linked to an “inducer-dependent promoter” or an “inducer-independent promoter”. Further, the polynucleotide encoding the third alanine racemase is, preferably, operably linked to an “inducer-independent promoter”, such as a constitutive promoter.

An “inducer dependent promoter” is understood herein as a promoter that is increased in its activity to enable transcription of the gene to which the promoter is operably linked upon addition of an “inducer molecule” to the fermentation medium. Thus, for an inducer-dependent promoter, the presence of the inducer molecule triggers via signal transduction an increase in expression of the gene operably linked to the promoter. The gene expression prior activation by the presence of the inducer molecule does not need to be absent, but can also be present at a low level of basal gene expression that is increased after addition of the inducer molecule. The “inducer molecule” is a molecule, the presence of which in the fermentation medium is capable of affecting an increase in expression of a gene by increasing the activity of an inducer-dependent promoter operably linked to the gene. Preferably, the inducer molecule is a carbohydrate or an analog thereof. In one embodiment, the inducer molecule is a secondary carbon source of the Bacillus cell. In the presence of a mixture of carbohydrates, cells selectively take up the carbon source that provide them with the most energy and growth advantage (primary carbon source). Simultaneously, they repress the various functions involved in the catabolismand uptake of the less preferred carbon sources (secondary carbon source). Typically, a primary carbon source for Bacillus is glucose and various other sugars and sugar derivates being used by Bacillus as secondary carbon sources. Secondary carbon sources include e.g. mannose or lactose without being restricted to these.

Examples of inducer dependent promoters are given in the table below by reference to the respective operon:

| | Regulator | Type | | |
|---|---|---|---|---|
| Operon | a) | b) | Inducer | Organism |
| sacPA | SacT | AT | sucrose | B. subtilis |
| sacB | SacY | AT | sucrose | B. subtilis |
| bgl PH | LicT | AT | β-glucosides | B. subtilis |
| licBCAH | LicR | A | oligo-β-glucosides | B. subtilis |
| levDEFG sacL | LevR | A | fructose | B. subtilis |
| mtIAD | MtIR | A | mannitol | B. subtilis |
| manPA-yjdF | ManR | A | mannose | B. subtilis |
| manR | ManR | A | mannose | B. subtilis |
| bgIFB bgIG | BgIG | AT | β-glucosides | E. coli |
| lacTEGF | LacT | AT | lactose | L. casei |
| lacZYA | lacI | R | Allolactose; IPTG | E. coli |
| | | | (Isopropyl β-D-1- | |
| | | | thiogalactopyranoside) | |
| araBAD | araC | AR | L-arabinose | E. coli |
| xylAB | XylR | R | Xylose | B. subtilis |

a: transcriptional regulator protein
b: A: activator
AT: antiterminator
R: repressor
AR: activator/repressor

In contrast thereto, the activity of promoters that do not depend on the presence of an inducer molecule (herein called ‘inducer-independent promoters’) are either constitutively active or can be increased regardless of the presence of an inducer molecule that is added to the fermentation medium.

Constitutive promoters are independent of other cellular regulating factors and transcription initiation is dependent on sigma factor A (sigA). The sigA-dependent promoters comprise the sigma factor A specific recognition sites ‘−35’-region and ‘−10’-region.

Preferably, the ‘inducer-independent promoter’ sequence is selected from the group consisting of constitutive promoters not limited to promoters Pveg, PlepA, PserA, PymdA, Pfba and derivatives thereof with different strength of gene expression (Guiziou et al, (2016): Nucleic Acids Res. 44(15), 7495-7508), the aprE promoter of Subtilisin encoding aprE gene of Bacilli, the bacteriophage SPO1 promoters P4, P5, P15 (WO15118126), the cryIIIA promoter from Bacillus thuringiensis (WO9425612), the amyQ promoter from Bacillus amyloliquefaciens, the amyL promoter and promoter variants from Bacillus licheniformis (U.S. Pat. No. 5,698,415) and combinations thereof, or active fragments or variants thereof, preferably a constitutive promoter sequence.

WO9102792 discloses the functionality of the aprE promoter of the alkaline protease gene for the large-scale production of subtilisin Carlsberg-type protease in Bacillus licheniformis and its production in a fermentation process.

The promoters of the Bacillus pumilus genes aprE1 and aprE2 encoding for Subtilisin proteases have been applied for the expression of recombinant protease and amylase in Bacillus pumilus (Küppers T, Wiechert W. Microb Cell Fact. 2014 Mar. 24; 13(1):46.). In particular, the PaprE1-III promoter variant comprising nucleotides nt-382 relative to the start ATG showed very high productivity compared to PaprE1-IV promoter variant (nt-357 relative to the start ATG).

An “aprE promoter”, “aprE-type promoter” or “aprE promoter sequence” is the nucleotide sequence (or parts or variants thereof) located upstream of an aprE gene, i.e., a gene coding for a Bacillus subtilisin Carlsberg protease, on the same strand as the aprE gene that enables that aprE gene's transcription.

The term “transcription start site” or “transcriptional start site” shall be understood as the location where the transcription starts at the 5′ end of a gene sequence. In prokaryotes the first nucleotide, referred to as +1 is in general an adenosine (A) or guanosine (G) nucleotide. In this context, the terms “sites” and “signal” can be used interchangeably herein.

Further optionally the promoter comprises a 5′UTR. This is a transcribed but not translated region downstream of the −1 promoter position. Such untranslated region for example should contain a ribosome binding site to facilitate translation in those cases where the target gene codes for a peptide or polypeptide.

WO9102792 discloses the functionality of the promoter of the alkaline protease gene for the large-scale production of subtilisin Carlsberg-type protease in Bacillus licheniformis. In particular, WO9102792 describes the 5′ region of the subtilisin Carlsberg protease encoding aprE gene of Bacillus licheniformis (FIG. 27) comprising the functional aprE gene promoter and the 5′UTR comprising the ribosome binding site (Shine Dalgarno sequence).

The term “expression” or “gene expression” means the transcription of a specific gene or specific genes or specific nucleic acid construct. The term “expression” or “gene expression” in particular means the transcription of a gene or genes or genetic construct into structural RNA (e.g., rRNA, tRNA) or mRNA with or without subsequent translation of the latter into a protein. The process includes transcription of DNA and processing of the resulting mRNA product.

Further optionally the promoter comprises a 5′UTR. This is a transcribed but not translated region downstream of the-1 promoter position. Such untranslated region for example should contain a ribosome binding site to facilitate translation in those cases where the target gene codes for a peptide or polypeptide.

With respect to the 5′UTR the invention in particular teaches to combine the promoter of the present invention with a 5′UTR comprising one or more stabilizing elements. This way the mRNAs synthesized from the promoter region may be processed to generate mRNA transcript with a stabilizer sequence at the 5′ end of the transcript. Preferably such a stabilizer sequence at the 5′end of the mRNA transcripts increases their half-life as described by Hue et al, 1995, Journal of Bacteriology 177: 3465-3471. Suitable mRNA stabilizing elements are those described in
WO08148575, preferably SEQ ID NO. 1 to 5 of WO08140615, or fragments of these sequences which maintain the mRNA stabilizing function, and in
WO08140615, preferably Bacillus thuringiensis CryIIIA mRNA stabilising sequence or bacteriophage SP82 mRNA stabilising sequence, more preferably a mRNA stabilising sequence according to SEQ ID NO. 4 or 5 of WO08140615, more preferably a modified mRNA stabilising sequence according to SEQ ID NO. 6 of WO08140615, or fragments of these sequences which maintain the mRNA stabilizing function.

Preferred mRNA stabilizing elements are selected from the group consisting of aprE, grpE, cotG, SP82, RSBgsiB, CryIIIA mRNA stabilizing elements, or according to fragments of these sequences which maintain the mRNA stabilizing function. A preferred mRNA stabilizing element is the grpE mRNA stabilizing element (corresponding to SEQ ID NO. 2 of WO08148575).

The 5′UTR also preferably comprises a modified rib leader sequence located downstream of the promoter and upstream of a ribosome binding site (RBS). In the context of the present invention a rib leader is herewith defined as the leader sequence upstream of the riboflavin biosynthetic genes (rib operon) in a Bacillus cell, more preferably in a Bacillus subtilis cell. In Bacillus subtilis, the rib operon, comprising the genes involved in riboflavin biosynthesis, include ribG (ribD), ribB (ribE), ribA, and ribH genes. Transcription of the riboflavin operon from the rib promoter (Prib) in B. subtilis is controlled by a riboswitch involving an untranslated regulatory leader region (the rib leader) of almost 300 nucleotides located in the 5′-region of the rib operon between the transcription start and the translation start codon of the first gene in the operon, ribG. Suitable rib leader sequences are described in WO2015/1181296, in particular pages 23-25, incorporated herein by reference.

For industrial fermentation processes, the bacterial host cell may be genetically modified to meet the needs of highest product purity and regulatory requirements. It is therefore in scope of the invention to use Bacillus production hosts that may additionally contain modifications, e.g., deletions or disruptions, of other genes that may be detrimental to the production, recovery or application of a polypeptide of interest. In one embodiment, a bacterial host cell is a protease-deficient cell. The bacterial host cell, e.g., Bacillus cell, preferably comprises a disruption or deletion of extracellular protease genes including but not limited to aprE, mpr, vpr, bpr, and/or epr. Further preferably the bacterial host cell does not produce spores. Further preferably the bacterial host cell, e.g., a Bacillus cell, comprises a disruption or deletion of genes involved in sporulation. Genes involved in sporulation are well known in the art (EP1391502), comprising but not limited to sigE, sigF, spolIGA, spolIE, sigG, spolVCB, yqfD. In a preferred embodiment, the sigF gene is deleted. Further, preferably the bacterial host cell, e.g., Bacillus cell, comprises a disruption or deletion of one of the genes involved in the biosynthesis of surfactin, e.g., srfA, srfB, srfC, and/or srfD, see, for example, U.S. Pat. No. 5,958,728. It is also preferred that the bacterial host cell comprises a disruption or deletion of one of the genes involved in the biosynthesis of polyglutamic acid (US2016002591). Other genes, including but not limited to the amyE gene, which are detrimental to the production, recovery or application of a polypeptide of interest may also be disrupted or deleted.

In one embodiment, the Bacillus cell comprises a selectable marker. The selectable marker can be antibiotic resistance markers such as ampicillin, kanamycin, erythromycin, chloramphenicol or tetracycline, or an auxotrophic resistance marker.

Optionally the Bacillus cell might comprise a counterselection markers as described herein. In a preferred embodiment, the counterselection polypeptide is a polypeptide which involved in the pyrimidine metabolism. Thus, the counterselection polypeptide, such as oroP, pyrE, pyrF, upp, uses flourated analogons of intermediates in the pyrimidine metabolism, such as, 5-fluoro-orotate or 5-fluoro-uridine. Alternatively, toxins of toxin-anti-toxin systems (TA) such as the mazEF, ccdAB could be used as functional counterselection polypeptides in Bacillus (see Dong, H., Zhang, D. Current development in genetic engineering strategies of Bacillus species. Microb Cell Fact 13, 63 (2014)). In an even more preferred embodiment, the couterselection polypeptide is a cytosine deaminase, such as provided by the codBA system (Kostner D, Rachinger M, Liebl W, Ehrenreich A. Markerless deletion of putative alanine dehydrogenase genes in Bacillus licheniformis using a codBA-based counterselection technique. Microbiology. 2017; 163(11): 1532-1539). Preferably, the counterselection agent is 5-fluoro-cytosine.

### The Compound of Interest

The host cell of the present invention shall further comprise an expression cassette for the production of a compound of interest, preferably a polypeptide of interest.

Compounds of interest maybe polymers, preferably hyaluronic acidy, preferably as described in (WO2005098016), or polyglutamic acid, preferably as described in EP2196534, or maybe vitamins, preferably vitamin B5, preferably as described in WO2010018169, or riboflavin, preferably as described in WO2017036903, or may be polypeptides, preferably enzymes.

The term “polypeptide of interest” as used herein refers to any protein, peptide or fragment thereof which is intended to be produced in the bacterial host cell. A protein, thus, encompasses polypeptides, peptides, fragments thereof as well as fusion proteins and the like.

Preferably, the compound of interest, preferably the polypeptide of interest, is secreted by the Bacillus licheniformis host cell out of the cell.

Preferably, the polypeptide of interest is an enzyme, such as an exoenzyme. An exoenzyme (or extracellular enzyme), is an enzyme that is secreted by the host cell.

In a particularly preferred embodiment, the enzyme is classified as an oxidoreductase (EC 1), a transferase (EC 2), a hydrolase (EC 3), a lyase (EC 4), an isomerase (EC 5), or a ligase (EC 6).

In a preferred embodiment, the protein of interest is an enzyme suitable to be used in detergents, feed, and food applications.

Most preferably, the enzyme is a hydrolase (EC 3), preferably, a glycosidase (EC 3.2) or a peptidase (EC 3.4). Especially preferred enzymes are enzymes selected from the group consisting of an amylase (in particular an alpha-amylase (EC 3.2.1.1), beta-beta amylase (EC 3.2.1.2), a cellulase (EC 3.2.1.4), an endo-1,3-beta-xylanase xylanase (EC 3.2.1.32), an endo-1,4-beta-xylanase (EC 3.2.1.8), a lactase (EC 3.2.1.108), a galactosidase (EC 3.2.1.23 and EC 3.2.1.24), a mannanase (EC 3.2.1.24 and EC 3.2.1.25), a lipase (EC 3.1.1.3), a phytase (EC 3.1.3.8), a nuclease (EC 3.1.11 to EC 3.1.31), and a protease (EC 3.4); in particular an enzyme selected from the group consisting of amylase, protease, lipase, mannanase, phytase, xylanase, phosphatase, ß-galactosidase, lactase glucoamylase, nuclease, and cellulase, preferably, amylase, mannanase, lactase or protease, preferably, an amylase and a protease. Most preferred is a serine protease (EC 3.4.21), preferably a subtilisin protease.

In particular, the following proteins of interest are preferred:

Enzymes having proteolytic activity are called “proteases” or “peptidases”. Proteases are active proteins exerting “protease activity” or “proteolytic activity”. Proteases are members of class EC 3.4. Proteases include aminopeptidases (EC 3.4.11), dipeptidases (EC 3.4.13), dipeptidyl-peptidases and tripeptidyl-peptidases (EC 3.4.14), peptidyl-dipeptidases (EC 3.4.15), serine-type carboxypeptidases (EC 3.4.16), metallocarboxypeptidases (EC 3.4.17), cysteine-type carboxypeptidases (EC 3.4.18), omega peptidases (EC 3.4.19), serine endopeptidases (EC 3.4.21), cysteine endopeptidases (EC 3.4.22), aspartic endopeptidases (EC 3.4.23), metallo-endopeptidases (EC 3.4.24), threonine endopeptidases (EC 3.4.25), endo-peptidases of unknown catalytic mechanism (EC 3.4.99). Commercially available protease enzymes include but are not limited to Lavergy™ Pro (BASF); Alcalase®, Blaze®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coro-nase®, Coronase® Ultra, Neutrase®, Everlase® and Esperase® (Novozymes A/S), those sold under the tradename Maxatase®, Maxacal®, Maxapem®, Pura-fect®, Purafect® Prime, Pura-fect MA®, Purafect Ox®, Purafect OxP®, Puramax®, Properase®, FN2®, FN3®, FN4®, Ex-cellase®, Eraser®, Ultimase®, Opticlean®, Effectenz®, Preferenz® and Optimase® (Dan-isco/DuPont), Axapem™ (Gist-Brocases N.V.), Bacillus lentus Alkaline Protease, and KAP (Bacillus alkalophilus subtilisin) from Kao. At least one protease may be selected from serine proteases (EC 3.4.21). Serine proteases or serine peptidases (EC 3.4.21) are characterized by having a serine in the catalytically active site, which forms a covalent adduct with the substrate during the catalytic reaction. A serine protease may be selected from the group consisting of chymotrypsin (e.g., EC 3.4.21.1), elastase (e.g., EC 3.4.21.36), elastase (e.g., EC 3.4.21.37 or EC 3.4.21.71), granzyme (e.g., EC 3.4.21.78 or EC 3.4.21.79), kallikrein (e.g., EC 3.4.21.34, EC 3.4.21.35, EC 3.4.21.118, or EC 3.4.21.119,) plasmin (e.g., EC 3.4.21.7), trypsin (e.g., EC 3.4.21.4), thrombin (e.g., EC 3.4.21.5,) and subtilisin (also known as subtilopeptidase, e.g., EC 3.4.21.62), the latter hereinafter also being referred to as “subtilisin”. Preferably, the protease is a protease variant of Bacillus lentus alkaline protease (BLAP), most preferably BLAP comprising the substitution R101E (according to BPN′ numbering). Proteases according to the invention have proteolytic activity. The methods for determining proteolytic activity are well-known in the literature (see e.g. Gupta et al. (2002), Appl. Microbiol. Bio-technol. 60: 381-395).

Thus, preferably, the present invention relates to a method for producing an enzyme, preferably a protease or an amylase, comprising
a) providing the modified Bacillus licheniformis host cell comprising at least one of
   a. a genetic modification causing compared to an unmodified control cell an increased expression of at least one of the genes selected from the group consisting of degQ, degU, degS, degR, and phrG,
   b. a genetic modification causing compared to an unmodified control cell an increased autophosphorylation activity of the DegS protein, and
   c. a genetic modification causing compared to an unmodified control cell a reduced phosphatase activity of the DegS protein,
b) introducing onto said modified Bacillus host cell an expression cassette for the enzyme, preferably the enzyme being secreted by the host cell,
c) cultivating the host cell under conditions which allow for the expression of the enzyme, and
d) optionally isolating the enzyme from the cultivation medium.

Preferably, the present invention relates to a method for producing an enzyme, preferably a protease or an amylase, comprising
a) providing the modified Bacillus licheniformis host cell comprising a genetic modification causing compared to an unmodified control cell an increased expression of at least one of the degQ gene,
b) introducing onto said modified Bacillus host cell an expression cassette for an enzyme, preferably an enzyme that is secreted by the host cell,
c) cultivating the host cell under conditions which allow for the expression of the enzyme, and
d) optionally isolating the compound of interest from the cultivation medium.

Preferably, the present invention relates to a method for producing an enzyme, preferably a protease or an amylase, comprising
a) providing the modified Bacillus licheniformis host cell comprising
   a. a genetic modification causing compared to an unmodified control cell an increased expression of at least one of the degQ gene, and optionally
   b. a genetic modification causing compared to an unmodified control cell an increased autophosphorylation activity of the DegS protein, or
   c. a genetic modification causing compared to an unmodified control cell a reduced phosphatase activity of the DegS protein,
b) introducing onto said modified Bacillus host cell an expression cassette for an enzyme, preferably an enzyme that is secreted by the host cell,
c) cultivating the host cell under conditions which allow for the expression of the enzyme, and
d) optionally isolating the enzyme from the cultivation medium.

### Preferred Embodiments

1. A modified Bacillus licheniformis host cell comprising at least one of
   a) a genetic modification causing compared to an unmodified control cell an increased expression of at least one of the genes selected from the group consisting of degQ, degU, degS, degR, and phrG,
   b) a genetic modification causing compared to an unmodified control cell an increased autophosphorylation activity of the DegS protein, and
   c) a genetic modification causing compared to an unmodified control cell a reduced phosphatase activity of the DegS protein.
2. The modified Bacillus licheniformis host cell of embodiment 1, wherein the modification of any of a) to c) results in an increased level of phosphorylated DegU protein in the Bacillus licheniformis host cell compared to an unmodified control cell.
3. The modified Bacillus licheniformis host cell of any of the preceding embodiments, wherein the Bacillus licheniformis host cell comprises a reduced expression of the Formosin D protein compared to an unmodified control cell, preferably, wherein the Formosin D protein comprises with increasing preference a sequence having at least 80%, at least 90%, at least 95%, at least 99%, or 100% identity to SEQ ID NO: 34, preferably, the modified Bacillus licheniformis host cell comprises no modification in the endogenous forD gene.
4. The modified Bacillus licheniformis host cell of any of the preceding embodiments, wherein the Bacillus licheniformis host cell comprises a genetic modification causing compared to an unmodified control cell an increased expression of at least one of the genes selected from the group consisting of degQ, degU, degS, and degR, preferably, an increased expression of the degQ gene.
5. The modified Bacillus licheniformis host cell of any of the preceding embodiments, wherein the Bacillus licheniformis host cell comprises a genetic modification causing compared to an unmodified control cell an increased expression of the degQ gene.
6. The modified Bacillus licheniformis host cell of any of the preceding embodiments, wherein the Bacillus licheniformis host cell comprises a genetic modification causing compared to an unmodified control cell an increased expression of the degQ gene and wherein the Bacillus licheniformis host cell comprises a genetic modification causing compared to an unmodified control cell an increased autophosphorylation activity of the DegS protein or a genetic modification causing compared to an unmodified control cell a reduced phosphatase activity of the DegS protein.
7. The modified Bacillus licheniformis host cell of any of the preceding embodiments, wherein the genetic modification causing an increased autophosphorylation activity of DegS leads to the amino acid substitution X76D, preferably S76D, in the DegS protein (according to the numbering of SEQ ID NO: 41) of the Bacillus licheniformis host cell.
8. The modified Bacillus licheniformis host cell of any of the preceding embodiments, wherein the genetic modification causing a reduced phosphatase activity of DegS leads to the amino acid substitution X218E, preferably G218E, (according to the numbering of SEQ ID NO: 41) in the DegS protein of the Bacillus licheniformis host cell.
9. The modified Bacillus licheniformis host cell of any of the preceding embodiments, wherein the degU gene encodes a DegU protein comprising an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 54, wherein the degS gene encodes a DegS protein comprising an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 41, wherein the degQ gene encodes a DegQ protein comprising an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 37, wherein the degR gene encodes a DegR protein comprising an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 58, and wherein the phrG gene encodes a PhrG protein comprising an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 62.
10. The modified Bacillus licheniformis host cell of any of the preceding embodiments, wherein the degQ gene encodes a DegQ protein comprising an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 37.
11. The modified Bacillus licheniformis host cell of any of the preceding embodiments, wherein the degQ gene is increased in expression by introducing into the host cell an expression construct comprising a gene coding for the DegQ protein, preferably comprising a nucleotide sequence having at least 80%, at least 90%, or 100% to nucleotides 11-151 of SEQ ID NO: 4, operably linked to promoter sequence, preferably comprising a nucleotide sequence having at least 80%, at least 90%, or 100% to nucleotides 19-94 of SEQ ID NO: 3, preferably, the expression construct is integrated as an additional copy of the degQ gene into the chromosome of the Bacillus licheniformis host cell.
12. The modified Bacillus host cell of any one of the preceding embodiments, wherein the host cell comprises an expression cassette for the production of a compound of interest, preferably a polypeptide of interest.
13. The modified Bacillus host cell of embodiment 12, wherein the polypeptide of interest is an enzyme, such as an enzyme selected from the group consisting of amylase, protease, lipase, phospholipase, mannanase, phytase, xylanase, lactase, phosphatase, glucoamylase, nuclease, galactosidase, endoglucanase and cellulase.
14. The modified Bacillus licheniformis host cell of any of embodiments 11-13, wherein the protein of interest comprises a signal sequence for secretion of the polypeptide of interest out of the host cell.
15. The modified Bacillus licheniformis host cell of any of embodiments 11-14, wherein the Bacillus licheniformis host cell produces the compound of interest with an increased purity, preferably with a reduced contamination with the Formosin D protein.
16. The modified Bacillus licheniformis host cell of any of embodiments 11-15, wherein the Bacillus licheniformis host cell comprises an increased production of polypeptide of interest compared to an unmodified Bacillus licheniformis control cell.
17. A method for producing a compound of interest, preferably a polypeptide of interest, comprising
   a) providing a modified Bacillus licheniformis host cell of any of embodiments 11-16,
   b) cultivating the host cell under conditions which allow for the expression of the compound of interest, and
   c) optionally isolating the compound of interest from the cultivation medium.
18. A method for increasing the purity of a compound of interest, preferably a polypeptide of interest, produced by a Bacillus licheniformis cell, comprising the steps of
   a) providing a modified Bacillus licheniformis host cell of any of embodiments 11-16,
   b) cultivating the host cell under conditions which allow for the expression of the compound of interest, and
   c) isolating the compound of interest from the cultivation medium.
19. The method of embodiment 17 or 18, wherein the Bacillus licheniformis host cell comprises a reduced expression of the Formosin D protein compared to an unmodified control cell, preferably, wherein the Formosin D protein comprises with increasing preference a sequence having at least 80%, at least 90%, at least 95%, at least 99%, or 100% identity to SEQ ID NO: 34.
20. A method for reducing the Formosin D protein contamination of a compound of interest, preferably a polypeptide of interest, produced by a Bacillus licheniformis cell, comprising the steps of
   a) providing a modified Bacillus licheniformis host cell of any of claims 11-16,
   b) cultivating the host cell under conditions which allow for the expression of the compound of interest, and
   c) isolating the compound of interest from the cultivation medium.
21. The modified Bacillus licheniformis host cell and method of any of the preceding embodiments, wherein the control cell is a Bacillus licheniformis cell, which differs from the host cell only in that it does not carry the respective modification.

### EXAMPLES

### Material and Methods

The following examples only serve to illustrate the invention. The numerous possible variations that are obvious to a person skilled in the art also fall within the scope of the invention. Unless otherwise stated the following experiments have been performed by applying standard equipment, methods, chemicals, and biochemicals as used in genetic engineering, molecular biology and fermentative production of chemical compounds by cultivation of microorganisms. See also Sambrook et al. (Sambrook, J. and Russell, D. W. Molecular cloning. A laboratory manual, 3rd ed, Cold Spring Harbor Laboratory Press, Cold Spring Harbor, N Y. 2001) and Chmiel et al. (Bioprocesstechnik 1. Einführung in die Bioverfahrenstechnik, Gustav Fischer Verlag, Stuttgart, 1991).

Electrocompetent Bacillus licheniformis Cells and Electroporation

Transformation of DNA into Bacillus licheniformis strain DSM641 U.S. Pat. No. 5,352,604 is performed via electroporation. Preparation of electrocompetent Bacillus licheniformis cells and transformation of DNA is performed as essentially described by Brigidi et al (Brigidi, P., Mateuzzi, D. (1991). Biotechnol. Techniques 5, 5) with the following modification: Upon transformation of DNA, cells are recovered in 1 ml LBSPG buffer and incubated for 60 min at 37° C. (Vehmaanperä J., 1989, FEMS Microbio. Lett., 61: 165-170) following plating on selective LB-agar plates.

### Plasmid Isolation

Plasmid DNA was isolated from Bacillus and E. coli cells by standard molecular biology methods described in (Sambrook, J. and Russell, D. W. Molecular cloning. A laboratory manual, 3rd ed, Cold Spring Harbor Laboratory Press, Cold Spring Harbor, N Y. 2001) or the alkaline lysis method (Birnboim, H. C., Doly, J. (1979). Nucleic Acids Res 7(6): 1513-1523). Bacillus cells were in comparison to E. coli treated with 10 mg/ml lysozyme for 30 min at 37° C. prior to cell lysis.

### Plasmids

Plasmid p689-T2A-lac

The E. coli plasmid p689-T2A-lac which is a derivative of the pUC57 vector comprises the lacZ-alpha gene flanked by BpiI restriction sites, again flanked 5′ by the T1 terminator of the E. coli rrnB gene and 3′ by the TO lambda terminator which was ordered as gene synthesis construct (SEQ ID 027).

pEC194RS—Bacillus Temperature Sensitive Deletion Plasmid.

The plasmid pE194 is PCR-amplified with oligonucleotides SEQ ID 009 and SEQ ID 010 with flanking PvuII sites, digested with restriction endonuclease PvuII and ligated into vector pCE1 digested with restriction enzyme SmaI. pCE1 is a pUC18 derivative, where the BsaI site within the ampicillin resistance gene has been removed by a silent mutation. The ligation mixture was transformed into E. coli DH10B cells (Life technologies). Transformants were spread and incubated overnight at 37° C. on LB-agar plates containing 100 μg/ml ampicillin. Plasmid DNA was isolated from individual clones and analyzed for correctness by restriction digest. The resulting plasmid is named pEC194S.

The type-II-assembly mRFP cassette is PCR-amplified from plasmid pBSd141R (accession number: KY995200) (Radeck, J., Mascher, T. 2017. Bacillus SEVA siblings: A Golden Gate-based toolbox to create personalized integrative vectors for Bacillus subtilis. Sci. Rep. 7: 14134) with oligonucleotides SEQ ID 011 and SEQ ID 012, comprising additional nucleotides for the restriction site BamHI. The PCR fragment and pEC194S were restricted with restriction enzyme BamHI following ligation and transformation into E. coli DH10B cells (Life technologies). Transformants were spread and incubated overnight at 37° C. on LB-agar plates containing 100 μg/ml ampicillin. Plasmid DNA was isolated from individual clones and analyzed for correctness by restriction digest. The resulting plasmid pEC194RS carries the mRFP cassette with the open reading frame opposite to the reading frame of the erythromycin resistance gene.

pDel003—aprE Gene Deletion Plasmid

The gene deletion plasmid for the aprE gene of Bacillus licheniformis was constructed with plasmid pEC194RS and the gene synthesis construct SEQ ID 021 comprising the genomic regions 5′ and 3′ of the aprE gene flanked by BsaI sites compatible to pEC194RS. The type-II-assembly with restriction endonuclease BsaI was performed as described (Radeck et al., 2017; Sci. Rep. 7: 14134) and the reaction mixture subsequently transformed into E. coli DH10B cells (Life technologies). Transformants were spread and incubated overnight at 37° C. on LB-agar plates containing 100 μg/ml ampicillin. Plasmid DNA was isolated from individual clones and analyzed for correctness by restriction digest. The resulting aprE deletion plasmid is named pDel003.

pDel005—sigF Gene Deletion Plasmid The gene deletion plasmid for the sigF gene (spollAC gene) of Bacillus licheniformis was constructed as described for pDel003, however the gene synthesis construct SEQ ID 024 comprising the genomic regions 5′ and 3′ of the sigF gene flanked by BsaI sites compatible to pEC194RS was used. The resulting sigF deletion plasmid is named pDel005.
pDel006—Restrictase Gene Deletion Plasmid

The gene deletion plasmid for the restrictase gene (SEQ ID 014) of the restriction modification system of Bacillus licheniformis DSM641 U.S. Pat. No. 5,352,604 (SEQ ID 013) was constructed with plasmid pEC194RS and the gene synthesis construct SEQ ID 015 comprising the genomic regions 5′ and 3′ of the restrictase gene flanked by BsaI sites compatible to pEC194RS. The type-II-assembly with restriction endonuclease BsaI was performed as described above and the reaction mixture subsequently transformed into E. coli DH10B cells (Life technologies). Transformants were spread and incubated overnight at 37° C. on LB-agar plates containing 100 μg/ml ampicillin. Plasmid DNA was isolated from individual clones and analyzed for correctness by restriction digest. The resulting restrictase deletion plasmid is named pDel006.

pDel007—Poly-Gamma-Glutamate Synthesis Genes Deletion Plasmid

The deletion plasmid for deletion of the genes involved in poly-gamma-glutamate (pga) production, namely ywsC (pgsB), ywtA (pgsC), ywtB (pgsA), ywtC (pgsE) of Bacillus licheniformis was constructed as described for pDel006, however the gene synthesis construct SEQ ID 018 comprising the genomic regions 5′ and 3′ flanking the ywsC (pgsB), ywtA (pgsC), ywtB (pgsA), ywtC (pgsE) genes flanked by BsaI sites compatible to pEC194RS was used. The resulting pga deletion plasmid is named pDel007.

pDel023—Formosin D Deletion Plasmid

The gene deletion plasmid for the formosin D gene (forD gene) of Bacillus licheniformis was constructed as described for pDel003, however the gene synthesis construct SEQ ID 030 comprising the genomic regions 5′ and 3′ of the forD gene with mutated RBS sequence flanked by BsaI sites compatible to pEC194RS was used. The resulting forD deletion plasmid is named pDel023.

pMA110—Pga::BLAP Integration Plasmid

The pga::BLAP integration plasmid for integration of the BLAP gene expression cassette into the poly-gamma-glutamate synthesis locus was constructed by a two-step cloning strategy. The BLAP protease expression cassette comprising the PaprE promoter was PCR amplified from pCB56C (U.S. Pat. No. 5,352,604) using oligonucleotides Seq ID 028 and Seq ID 029 and subcloned into p689-T2A-lac in a type-II-assembly reaction with restriction endonuclease BpiI. The resulting plasmid p689-BLAP was sequence verified. The plasmid for exchange of the pga synthesis locus by the BLAP expression cassette was constructed in a second type-II-assembly reaction with restriction endonuclease BsaI with plasmids pEC194RS and p689-BLAP, and 5′ and 3′ homology regions of the pga synthesis locus (Seq ID 001 and Seq ID 02) provided as gene synthesis constructs flanked by BsaI restriction sites compatible with pEC194RS and p689-BLAP to allow for directed cloning. The resulting sequence verified plasmid was named pMA110.

pInt010—CAT::Psy-degQ Integration Plasmid

The CAT::Psy-degQ integration plasmid for integration of the Bacillus licheniformis degQ gene expression cassette into the chloramphenicol-acetyltransferase (CAT) locus was constructed by a two-step cloning strategy. The degQ expression cassette comprising a synthetic promoter variant of the Bacillus subtilis Pveg promoter referred to as Psy (SEQ ID 03) and the degQ gene fragment of Bacillus licheniformis (SEQ ID 04) were ordered as gene synthesis fragments with flanking BpiI restriction endonuclease sites and subcloned into p689-T2A-lac in a type-II-assembly reaction with restriction endonuclease BpiI. The resulting plasmid p689-Psy-degQ was sequence verified. The plasmid for exchange of the CAT locus by the degQ expression cassette was constructed in a second type-II-assembly reaction with restriction endonuclease BsaI with plasmids pEC194RS and p689-Psy-degQ, and 5′ and 3′ homology regions of the CAT locus (Seq ID 05 and Seq ID 06) provided as gene synthesis constructs flanked by BsaI restriction sites compatible with pEC194RS and p689-Psy-degQ to allow for directed cloning. The resulting sequence-verified plasmid was named pInt010.

### Strains

E. coli Strain Ec #098

E. coli strain Ec #098 is an E. coli INV110 strain (Life technologies) carrying the DNA-methyltransferase encoding expression plasmid pMDS003 WO2019016051.

Generation of Bacillus licheniformis Gene k.o. Strains

For gene deletion in Bacillus licheniformis strains DSM641 (U.S. Pat. No. 5,352,604) and derivatives thereof deletion plasmids were transformed into E. coli strain Ec #098 made competent according to the method of Chung (Chung, C. T., Niemela, S. L., and Miller, R. H. (1989). One-step preparation of competent Escherichia coli: transformation and storage of bacterial cells in the same solution. Proc. Natl. Acad. Sci. U.S.A 86, 2172-2175), following selection on LB-agar plates containing 100 μg/ml ampicillin and 30 μg/ml chloramphenicol at 37° C. Plasmid DNA was isolated from individual clones and used for subsequent transfer into Bacillus licheniformis strains. The isolated plasmid DNA carries the DNA methylation pattern of Bacillus licheniformis strains DSM641 respectively and is protected from degradation upon transfer into Bacillus licheniformis. For gene deletion in Bacillus licheniformis strains with deleted restrictase gene, deletion plasmids were constructed within E. coli INV110 cells (Life technologies).

Bacillus licheniformis P304: Deleted Restriction Endonuclease

Electrocompetent Bacillus licheniformis DSM641 cells (U.S. Pat. No. 5,352,604) were prepared as described above and transformed with 1 μg of pDel006 restrictase gene deletion plasmid isolated from E. coli Ec #098 following plating on LB-agar plates containing 5 μg/ml erythromycin at 30° C.

The gene deletion procedure was performed as described in the following:

Plasmid carrying Bacillus licheniformis cells were grown on LB-agar plates with 5 μg/ml erythromycin at 45° C. driving integration of the deletion plasmid via Campbell recombination into the chromosome with one of the homology regions of pDel006 homologous to the sequences 5′ or 3′ of the restrictase gene. Clones were picked and cultivated in LB-media without selection pressure at 45° C. for 6 hours, following plating on LB-agar plates with 5 μg/ml erythromycin and incubation overnight at 30° C. Individual clones were picked and screened by colony-PCR analysis with oligonucleotides SEQ ID 016 and SEQ ID 017 for successful genomic deletion of the restrictase gene. Putative deletion positive individual clones were picked and taken through two consecutive overnight incubation in LB media without antibiotics at 45° C. to cure the plasmid and plated on LB-agar plates for overnight incubation at 37° C. Single clones were analyzed by colony PCR for successful genomic deletion of the restrictase gene. A single erythromycin-sensitive clone with the correct deleted restrictase gene was isolated and designated Bacillus licheniformis P304.

Bacillus licheniformis P305: Deleted sigF Gene

Electrocompetent Bacillus licheniformis P304 cells were prepared as described above and transformed with 1 μg of pDel005 sigF gene deletion plasmid isolated from E. coli INV110 cells (Life technologies) following plating on LB-agar plates containing 5 μg/ml erythromycin and incubation overnight at 30° C.

The gene deletion procedure was performed as described for the restrictase gene.

The deletion of the sigF gene was analyzed by PCR with oligonucleotides SEQ ID 025 and SEQ ID 026. The resulting Bacillus licheniformis strain with a deleted sigF gene is designated Bacillus licheniformis P305 and is no longer able to sporulate as described (WO9703185).

Bacillus licheniformis P307: Deleted aprE Gene

Electrocompetent Bacillus licheniformis P305 cells were prepared as described above and transformed with 1 μg of pDel003 aprE gene deletion plasmid isolated from E. coli INV110 cells following plating on LB-agar plates containing 5 μg/ml erythromycin and incubation overnight at 30° C.

The gene deletion procedure was performed as described for the deletion of the restrictase gene. The deletion of the aprE gene was analyzed by PCR with oligonucleotides SEQ ID 022 and SEQ ID 023 The resulting Bacillus licheniformis strain with deleted aprE gene was named Bacillus licheniformis P307.

Bacillus licheniformis M309: Deleted Poly-Gamma Glutamate Synthesis Genes

Electrocompetent Bacillus licheniformis P307 cells were prepared as described above and transformed with 1 μg of pDel007 pga gene deletion plasmid isolated from E. coli INV110 cells (Life technologies) following plating on LB-agar plates containing 5 μg/ml erythromycin and incubation overnight at 30° C.

The gene deletion procedure was performed as described for the deletion of the restrictase gene. The deletion of the pga genes was analyzed by PCR with oligonucleotides SEQ ID 019 and SEQ ID 020 The resulting Bacillus licheniformis strain with deleted pga synthesis genes was named Bacillus licheniformis M309.

Bacillus licheniformis P311: Deleted forD Gene

Electrocompetent Bacillus licheniformis M309 cells were prepared as described above and transformed with 1 μg of pDel023 forD gene deletion plasmid isolated from E. coli INV110 cells following plating on LB-agar plates containing 5 μg/ml erythromycin and incubation overnight at 30° C.

The gene deletion procedure was performed as described for the deletion of the restrictase gene. The deletion of the forD gene was analyzed by PCR with oligonucleotides SEQ ID 031 and SEQ ID 032 The resulting Bacillus licheniformis strain with deleted forD gene was named Bacillus licheniformis P311.

Bacillus licheniformis M409: Integration of the BLAP Protease Expression Cassette into the Poly-Gamma Glutamate Synthesis (Pga) Locus

Electrocompetent Bacillus licheniformis P307 cells were prepared as described above and transformed with 1 μg of pMA110-pga::PaprE-BLAP gene deletion/integration plasmid isolated from E. coli INV110 cells (Life technologies) following plating on LB-agar plates containing 5 μg/ml erythromycin and incubation overnight at 30° C. (BLAP=Bacillus lentus alkaline protease). The gene deletion/integration procedure was performed as described for the deletion of the restrictase gene. The integration of the BLAP expression cassette by replacement of the pga genes was analyzed by PCR with oligonucleotides SEQ ID 019 and SEQ ID 020. The resulting Bacillus licheniformis strain with integrated BLAP expression cassette gene was named Bacillus licheniformis M409.

Bacillus licheniformis P312: Integration of the degQ Expression Cassette into the CAT Locus

Electrocompetent Bacillus licheniformis M309 cells were prepared as described above and transformed with 1 μg of pInt010 gene deletion/integration plasmid isolated from E. coli INV110 cells (Life technologies) following plating on LB-agar plates containing 5 μg/ml erythromycin and incubation overnight at 30° C.

The gene deletion/integration procedure was performed as described for the deletion of the restrictase gene. The integration of the degQ expression cassette by replacement of the CAT-locus was analyzed by PCR with oligonucleotides SEQ ID 007 and 008. The resulting Bacillus licheniformis strain with integrated degQ expression cassette gene was named Bacillus licheniformis P312.

Bacillus licheniformis P313: Deleted forD Gene

Electrocompetent Bacillus licheniformis M409 cells were prepared as described above and transformed with 1 μg of pDel023 forD gene deletion plasmid isolated from E. coli INV110 cells following plating on LB-agar plates containing 5 μg/ml erythromycin and incubation overnight at 30° C.

The gene deletion procedure was performed as described for the deletion of the restrictase gene. The deletion of the forD gene was analyzed by PCR with oligonucleotides SEQ ID 031 and SEQ ID 032 The resulting Bacillus licheniformis strain with deleted forD gene was named Bacillus licheniformis P313

Bacillus licheniformis PC57: Integration of the degQ Expression Cassette into the CAT Locus

Electrocompetent Bacillus licheniformis M409 cells were prepared as described above and transformed with 1 μg of pInt010 gene deletion/integration plasmid isolated from E. coli INV110 cells (Life technologies) following plating on LB-agar plates containing 5 μg/ml erythromycin and incubation overnight at 30° C.

The gene deletion/integration procedure was performed as described for the deletion of the restrictase gene. The integration of the degQ expression cassette by replacement of the CAT-locus was analyzed by PCR with oligonucleotides SEQ ID 007 and 008. The resulting Bacillus licheniformis strain with integrated degQ expression cassette gene was named Bacillus licheniformis PC57.

TABLE 3
Genes of Bacillus licheniformis and Bacillus subtilis

| | | B. licheniformis | B. subtilis |
|---|---|---|---|
| Gene name | Function | DNA | DNA |
| forD | Formosin D (bacteriocin) | SEQ ID 033 | n.a. |
| degQ | Pleiotropic regulator | SEQ ID 035 | SEQ ID 036 |
| degS | Two-component sensor | SEQ ID 039 | SEQ ID 040 |
| | kinase | | |

TABLE 4
Proteins of Bacillus licheniformis
and Bacillus subtilis (PRT: Protein)

| | | B. | B. |
|---|---|---|---|
| | | licheniformis | subtilis |
| Protein Name | Function | PRT | PRT |
| ForD | Formosin D (bacteriocin) | SEQ ID 034 | n.a |
| DegQ | Pleiotropic regulator | SEQ ID 037 | SEQ ID 038 |
| DegS | Two-component sensor | SEQ ID 041 | SEQ ID 042 |
| | kinase | | |
| DegS_S76D | Two-component sensor | SEQ ID 043 | SEQ ID 044 |
| | kinase (mutant) | | |
| DegS_G218E | Two-component sensor | SEQ ID 045 | SEQ ID 046 |
| | kinase (mutant) | | |

### Example 1

Bacillus licheniformis strains with genetic modifications (as indicated in Table 1) were cultivated in a microtiter plate-based fed-batch process (Habicher et al., 2019 Biotechnol J.; 15(2).

TABLE 1
Bacillus licheniformis mutant strains

| Bacillus licheniformis strain name | Genotype |
|---|---|
| DSM641 (P300) | Wild type |
| M309 | P300 DRe, DaprE, DsigF, Dpga |
| P311 | M309 DforD |
| P312 | M309 CAT::Psy-DegQ |

‘D’ denotes deleted;
RE: restriction endonuclease

All cultivations were conducted in an orbital shaker with a diameter of 25 mm (Innova 42, New Brunswick Scientific, Eppendorf AG; Hamburg, Germany) at 30° C. and 400 rpm. Strains were cultivated in two subsequent precultures in FlowerPlates (MTP-48-OFF, m2p-labs GmbH) for synchronization of growth. The first preculture was carried out in 800 μl TB medium inoculated with a fresh single colony from the strain streaked onto LB agar plates. After 20 h, the second preculture containing 800 μl V3 minimal medium (Meissner et al., 2015, Journal of industrial microbiology & biotechnology 42 (9): 1203-1215) as inoculated with 8 μl of the first preculture and cultivated for 24 h. Microtiter plate-based fed-batch main cultivations were conducted using 48-well round- and deep-well-microtiter plates with glucose-containing polymer on the bottom of each well (FeedPlate, article number: SMFP08004, Kuhner Shaker GmbH; Herzogenrath, Germany). 70 μl of the second preculture were used to inoculate 700 μl V3 minimal medium without glucose. Main cultures were incubated for 72 h. Precultures were covered with a sterile gas-permeable sealing foil (AeraSeal film, Sigma-Aldrich) to avoid contamination. FeedPlates were sealed with a sterile gas-permeable, evaporation reducing foil (F-GPR48-10, m2p-labs GmbH) to reduce evaporation and to avoid contamination.

At the end of the cultivation process, supernatants of cultivations were prepared by centrifugation and sterile filtration with a 0.2 μm filter. For SDS-PAGE analysis equal amounts of supernatants were directly resuspended with 2×SDS sample buffer and boiled for 10 min at 95° C., following loading on SDS-PAGE gel.

For SDS PAGE analysis a 4-12% BIS-TRIS-Gel (NuPAGE), 10 Well was used with MES Running Buffer. Staining was performed using Comassie Brillant Blue G250 dissolved in acetic acid/ethanol with destaining with performed with acetic acid/ethanol.

FIG. 1 shows the SDS PAGE of supernatants of cultivations of Bacillus licheniformis M309 strain in comparison to Bacillus licheniformis P311 strain with deleted forD gene, and Bacillus licheniformis P312 strain comprising the forD gene with an additional copy of the degQ gene under control of a constitutive promoter with increased DegQ expression levels. Surprisingly, in Bacillus licheniformis P312 strain the amount of Formosin D is strongly reduced compared to the control strain M309. As control, Bacillus licheniformis P311 strain with deleted forD gene does not show the Formosin D band on the SDS-PAGE. FIG. 1: SDS-PAGE of supernatant from B. licheniformis strains after 72 h of simulated fed-batch cultivation. 1-3=control strain; 4=ForD deletion strain; 5-7=DegQ overexpression strain; M=Precision Plus Protein Standard with indicated size in kDa of selected bands; filled arrowhead highlights the ForD protein band.

### Example 2

Bacillus licheniformis strains with genetic modifications (as indicated in Table 2) were cultivated in a microtiter plate-based fed-batch process as described in Example 1.

TABLE 2
Bacillus licheniformis mutant strains

| Bacillus licheniformis strain name | Genotype |
|---|---|
| DSM641 (P300) | Wild type |
| M309 | P300 DRe, DaprE, DsigF, Dpga |
| M409 | M309 pga::BLAP |
| P313 | M409 DforD |
| PC57 | M409 CAT::Psy-DegQ |

‘D’ denotes deleted;
RE: restriction endonuclease

At the end of the cultivation process, supernatants of cultivations were prepared by centrifugation and sterile filtration with a 0.2 μm filter. For SDS-PAGE analysis supernatants were TCA precipitated followed by SDS-PAGE with Coomassie Blue Staining. For TCA precipitation the sample was adjusted to 13.3% TCA (w/w), kept on ice for 10 min, then sedimented using a table top centrifuge, the pellet washed using aceton. The pellet was then resuspended using 1×SDS sample buffer. Prior SDS-PAGE the samples were boiled for 10 min at 95° C. For SDS-PAGE analysis equal amounts of supernatants were loaded onto the SDS-PAGE gel. For SDS PAGE analysis a 4-12% BIS-TRIS-Gel (NuPAGE), 10 Well was used with MES Running Buffer. Staining was performed using Comassie Brillant Blue G250 dissolved in acetic acid/ethanol with destaining with performed with acetic acid/ethanol.

FIG. 2 shows the SDS PAGE of supernatants of cultivations of Bacillus licheniformis M409 strain in comparison to Bacillus licheniformis P313 strain with deleted forD gene, and Bacillus licheniformis PC57 strain comprising the forD gene with an additional copy of the degQ gene under control of a constitutive promoter with increased DegQ expression levels. Surprisingly, in Bacillus licheniformis PC57 strain the amount of Formosin D is strongly reduced compared to the control strain M409. As control, Bacillus licheniformis P313 strain with deleted forD gene does not show the Formosin D band on the SDS-PAGE. FIG. 2: SDS-PAGE of supernatant from B. licheniformis protease expression strains after 72 h of simulated fed-batch cultivation. 1-3=control strain; 4=ForD deletion strain; 5-7=DegQ overexpression strain; M=Precision Plus Protein Standard with indicated size in kDa of selected bands; filled arrowhead highlights the ForD protein band; open arrowhead indicates the heterologous protease.

1. A modified Bacillus licheniformis host cell comprising at least one of
a) a genetic modification causing compared to an unmodified control cell an increased expression of at least one of the genes selected from the group consisting of degQ, degU, degS, degR, and phrG,
b) a genetic modification causing compared to an unmodified control cell an increased autophosphorylation activity of the DegS protein, and
c) a genetic modification causing compared to an unmodified control cell a reduced phosphatase activity of the DegS protein.

2. The modified Bacillus licheniformis host cell of claim 1, wherein the modification of any of a) to c) results in an increased level of phosphorylated DegU protein in the Bacillus licheniformis host cell compared to an unmodified control cell.

3. The modified Bacillus licheniformis host cell of claim 1, wherein the Bacillus licheniformis host cell comprises a reduced expression of the Formosin D protein compared to an unmodified control cell.

4. The modified Bacillus licheniformis host cell of claim 1, wherein the Bacillus licheniformis host cell comprises a genetic modification causing compared to an unmodified control cell an increased expression of at least one of the genes selected from the group consisting of degQ, degU, degS, and degR.

5. The modified Bacillus licheniformis host cell of claim 1, wherein the Bacillus licheniformis host cell comprises a genetic modification causing compared to an unmodified control cell an increased expression of the degQ gene.

6. The modified Bacillus licheniformis host cell of claim 1, wherein the Bacillus licheniformis host cell comprises a genetic modification causing compared to an unmodified control cell an increased expression of the degQ gene and wherein the Bacillus licheniformis host cell comprises a genetic modification causing compared to an unmodified control cell an increased autophosphorylation activity of the DegS protein or a genetic modification causing compared to an unmodified control cell a reduced phosphatase activity of the DegS protein.

7. The modified Bacillus licheniformis host cell of claim 1, wherein the genetic modification causing an increased autophosphorylation activity of DegS leads to the amino acid substitution X76D (according to the numbering of SEQ ID NO: 41) in the DegS protein of the Bacillus licheniformis host cell.

8. The modified Bacillus licheniformis host cell of claim 1, wherein the genetic modification causing a reduced phosphatase activity of DegS leads to the amino acid substitution X218E (according to the numbering of SEQ ID NO: 41) in the DegS protein of the Bacillus licheniformis host cell.

9. The modified Bacillus licheniformis host cell of claim 1, wherein the degU gene encodes a DegU protein comprising an amino acid sequence with at least 80%, at least 90%, or 100% identity to SEQ ID NO: 54, wherein the degS gene encodes a DegS protein comprising an amino acid sequence with at least 80%, at least 90%, or 100% identity to SEQ ID NO: 41, wherein the degQ gene encodes a DegQ protein comprising an amino acid sequence with at least 80%, at least 90%, or 100% identity to SEQ ID NO: 37, wherein the degR gene encodes a DegR protein comprising an amino acid sequence with at least 80%, at least 90%, or 100% identity to SEQ ID NO: 58, and wherein the phrG gene encodes a PhrG protein comprising an amino acid sequence with at least 80%, at least 90%, or 100% identity to SEQ ID NO: 62.

10. The modified Bacillus licheniformis host cell of claim 1, wherein the degQ gene encodes a DegQ protein comprising an amino acid sequence with at least 80%, at least 90%, or 100% identity to SEQ ID NO: 37.

11. The modified Bacillus host cell of claim 1, wherein the host cell comprises an expression cassette for the production of a compound of interest.

12. The modified Bacillus host cell of claim 11, wherein the polypeptide of interest is an enzyme, such as an enzyme selected from the group consisting of amylase, protease, lipase, phospholipase, mannanase, phytase, xylanase, lactase, phosphatase, glucoamylase, nuclease, galactosidase, endoglucanase and cellulase.

13. The modified Bacillus licheniformis host cell of claim 11, wherein the Bacillus licheniformis host cell comprises an increased production of polypeptide of interest compared to an unmodified Bacillus licheniformis control cell.

14. A method for producing a compound of interest, comprising
a) providing a modified Bacillus licheniformis host cell of claim 11,
b) cultivating the host cell under conditions which allow for the expression of the compound of interest, and
c) optionally isolating the compound of interest from the cultivation medium.

15. A method for increasing the purity of a compound of interest, comprising the steps of
a) providing a modified Bacillus licheniformis host cell of claim 11,
b) cultivating the host cell under conditions which allow for the expression of the compound of interest, and
c) isolating the compound of interest from the cultivation medium.

16. The modified Bacillus licheniformis host cell of claim 1, wherein the host cell is a host cell overexpressing at least one of the genes selected from the group consisting of degQ, degU, degS, degR, and phrG.

17. The method of claim 14, wherein the host cell is a host cell overexpressing at least one of the genes selected from the group consisting of degQ, degU, degS, degR, and phrG.
